SEQUENCE LISTING (15 sequences)

SEQ ID NO: 1 - protein, 5 aa, synthetic construct
  REGION 1..5: note = Heavy chain CDR1
SYAIS

SEQ ID NO: 2 - protein, 17 aa, synthetic construct
  REGION 1..17: note = Heavy chain CDR1
GIIPIFGTTNYAQQFQG

SEQ ID NO: 3 - protein, 10 aa, synthetic construct
  REGION 1..10: note = Heavy chain CDR1
EAVAADWLDP

SEQ ID NO: 4 - protein, 12 aa, synthetic construct
  REGION 1..12: note = Heavy chain CDR1
RASQSVSSSYLA

SEQ ID NO: 5 - protein, 7 aa, synthetic construct
  REGION 1..7: note = Heavy chain CDR1
GASSRAT

SEQ ID NO: 6 - protein, 9 aa, synthetic construct
  REGION 1..9: note = Heavy chain CDR1
QQYGSSRFT

SEQ ID NO: 7 - protein, 119 aa, synthetic construct
  REGION 1..119: note = Heavy Chain Variable Region
QVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 8 - protein, 108 aa, synthetic construct
  REGION 1..108: note = Light Chain Variable Region
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIK

SEQ ID NO: 9 - protein, 18 aa, synthetic construct
  REGION 1..18: note = Linker
GSTSGSGKPGSGEGSTKG

SEQ ID NO: 10 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 11 - protein, 105 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAY

SEQ ID NO: 12 - protein, 27 aa, Homo sapiens
FWVLVVVGGVLACYSLLVTVAFIIFWV

SEQ ID NO: 13 - DNA, 2159 bp, Homo sapiens
  source 1..2159: mol_type = genomic DNA
agtttcccacaagccaggctgatccttttctgtcagtccacttcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgacctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctcccca

SEQ ID NO: 14 - protein, 502 aa, synthetic construct
  REGION 1..502: note = Hu19-CD828Z chimeric antigen receptor
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 396 aa, Homo sapiens
MPNPRPGKPSAPSLALGPSPGASPSWRAAPKASDLLGARGPGGTFQGRDLRGGAHASSSSLNPMPPSQLQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

(19) United States
(12) Patent Application Publication
(10) Pub. No.: US 2026/0137718 A1
(43) Pub. Date: May 21, 2026

(54) METHODS FOR TREATING AUTOIMMUNE DISEASES

(71) Applicant: Kyverna Therapeutics, Inc., Emeryville, CA (US)

(72) Inventors: Dominique C. Borie, Palo Alto, CA (US); James Chung, Thousand Oaks, CA (US)

(21) Appl. No.: 19/402,188

(22) Filed: Nov. 26, 2025

Related U.S. Application Data
(63) Continuation of application No. 17/984,814, filed on Nov. 10, 2022, now abandoned.
(60) Provisional application No. 63/296,311, filed on Jan. 4, 2022.
(60) Provisional application No. 63/419,193, filed on Oct. 25, 2022.

(51) Int. Cl. A61K 35/17; A61K 38/17; A61P 19/02; A61P 37/06

(52) U.S. Cl. CPC A61K 35/17; A61K 38/1774; A61P 19/02; A61P 37/06

(57) ABSTRACT

The present document relates to methods and materials for treating a subject having an autoimmune disease.

### CROSS-REFERENCE TO RELATED APPLICATIONS

This application is a continuation of U.S. patent application Ser. No. 17/984,814, filed on Nov. 10, 2022, which claims priority to U.S. Provisional Patent Application No. 63/419,193, filed on Oct. 25, 2022, and U.S. Provisional Patent Application No. 63/296,311, filed on Jan. 4, 2022, the contents of each of which is incorporated herein by reference in its entirety.

### SEQUENCE LISTING

This application contains a Sequence Listing that has been submitted electronically as an XML file named 47902-0045001_SL_ST26.xml. The XML file, created on Nov. 10, 2022, is 21,307 bytes in size. The material in the XML file is hereby incorporated by reference in its entirety.

### TECHNICAL FIELD

The present disclosure generally relates to humanized chimeric antigen receptors (CARs) (e.g., anti-CD19 CARs) and their use in the treatment and/or prevention of autoimmune diseases.

### BACKGROUND

B cells express a wide array of cell surface molecules during their differentiation and proliferation. Most notable are the surface antigens CD19 and CD20.

T-cells have been genetically modified to express chimeric antigen receptors (CARs), which are fusion proteins comprised of an antigen recognition moiety and T-cell activation domains (see, e.g., Kershaw et al., supra, Eshhar et al., Proc. Natl. Acad. Sci. USA, 90 (2): 720-724 (1993), and Sadelain et al., Curr. Opin. Immunol., 21 (2): 215-223 (2009)). Anti-CD19 CARs have been used to successfully treat B cell cancers. See, U.S. Pat. No. 10,287,350.

Treatment of autoimmune diseases generally requires multiple treatments to control the disease. The most common treatments are corticosteroids and cytotoxic drugs, which can be very toxic. These drugs can also suppress the entire immune system, resulting in serious infection, and can have adverse side effects on bone marrow, liver, and/or kidneys. Thus, they do not easily lend themselves to repeated use (e.g., multiple treatments). Most patients develop severe manifestations of disease that may be life-threatening and require close monitoring and active treatment.

As mentioned, current treatment for B cell based autoimmune diseases includes high-dose glucocorticoids combined with immunosuppressants such as cyclophosphamide (CYC) or rituximab in order to induce remission and prevent further organ damage, however relapse is common and remains a significant clinical management challenge. Upon disease control, lower doses of glucocorticoids and a broader range of immunosuppressants such as azathioprine (AZA), methotrexate, and mycophenolate mofetil (MMF) are used to maintain disease control. Rituximab or CYC may be repeated upon relapse of disease activity. However, despite these treatments, significant unmet need remains for new therapies that can readily achieve sustained remission, effectively address the frequent relapses with existing therapies, and reduce the significant background immunosuppressive therapies required to maintain good disease control.

### SUMMARY

Provided herein are methods of treating B cell based autoimmune diseases not treatable or inadequately treated with conventional autoimmune treatments due to their toxicity profiles or limitations in their pharmacological action.

Also provided herein are compositions and methods of the instant invention that permit the elimination of B cells at tissue sites normally not accessible to other treatments.

Also provided herein are non-toxic humanized anti-CD19 CAR constructs that deplete B cells responsible for a patient's autoimmune disease. Being non-toxic allows for high doses of the anti-CD19 CAR construct and/or multiple doses which result in depletion of B cells at sites not treatable with conventional autoimmune treatments due to their toxicity profile.

Also provided herein are method of producing T cells, including T reg cells, T reg cells, and nucleic acid vectors useful for treating autoimmune disease.

Also, provided herein are methods of reducing the number of B cells in a subject having a B cell based autoimmune disease using anti-CD19 CARs (anti-CD19 CARs) that exhibit low levels of toxicities associated with anti-CD19 CAR therapy, including cytokine-release syndrome (CRS), and neurologic toxicities.

Also provided herein are methods of administration of anti-CD19 CARs comprising doses and time of administration to a patient suffering from a B cell based autoimmune disease.

In addition, the invention provides isolated or purified nucleic acid sequences encoding the foregoing CARs, vectors comprising such nucleic acid sequences, isolated T-cells comprising such vectors, and methods of destroying B-cells by contacting such isolated T-cells with a population of CD19-expressing B-cells in vivo

In some embodiments of any of the methods described herein, the anti-CD19 CAR comprises a humanized chimeric antigen receptor comprising an extracellular antigen-binding domain, a transmembrane domain, and an intracellular domain, wherein the intracellular domain comprises a cytoplasmic signaling domain and one or more co-stimulatory domains. See, U.S. Pat. No. 10,287,350.

In some embodiments of any of the methods described herein, the cytoplasmic signaling domain is a CD3 zeta intracellular signaling domain and the co-stimulatory domain comprises an intracellular signaling domain from at least one of 4-1BB, OX40, or CD28. In some embodiments of any of the methods described herein, the extracellular antigen-binding domain is an antibody or an antigen-binding fragment. In some embodiments of any of the methods described herein, the antigen-binding fragment is selected from the group consisting of a Fab, a F(ab′)₂ fragment, a scFv, a scAb, a dAb, and a single domain antibody. In some embodiments of any of the methods described herein, the extracellular antigen-binding domain comprises an scFv that is capable of binding to an antigen on an autoimmune cell. In some embodiments of any of the methods described herein, the antigen is CD19. In some embodiments of any of the methods described herein, the extracellular antigen-binding domain comprises: (i) a heavy chain variable domain comprising a CDR1, a CDR2, and a CDR3 of SEQ ID NOs: 1-3, respectively; and (ii) a light chain variable domain comprising a CDR1, a CDR2, and a CDR3 of SEQ ID NOs: 4-6, respectively. In some embodiments of any of the methods described herein, the antigen-binding fragment comprises: (i) a heavy chain variable domain that is at least 90% identical to SEQ ID NO: 7; and (ii) a light chain variable domain that is at least 90% identical to SEQ ID NO: 8.

In some embodiments of any of the methods described herein, the humanized chimeric antigen receptor comprises a sequence that is at least 90% identical to SEQ ID NO: 14. In some embodiments of any of the methods described herein, the humanized chimeric antigen receptor comprises a sequence that is at least 95% identical to SEQ ID NO: 14. In some embodiments of any of the methods described herein, the humanized chimeric antigen receptor comprises SEQ ID NO: 14.

In some embodiments of any of the methods described herein, the B cell related autoimmune diseases are hard to treat with conventional medicines, and comprise rheumatoid arthritis. In some embodiments of any of the methods described herein, the B cell related autoimmune disease comprises Sjogren's syndrome. In some embodiments of any of the methods described herein, the B cell related autoimmune disease comprises lapsed and/or refractory lupus nephritis. In some embodiments, the B cell related autoimmune disease comprises systemic lupus erythematosus. Other B cell related autoimmune diseases include dermatomyositis, polymyositis, diffuse cutaneous systemic sclerosis (dcSSc), and anti-neutrophil cytoplasmic antibodies (ANCA)-associated vasculitis (AAV).

In some embodiments of any of the methods described herein, the subject has previously been treated with a lymphodepletion agent. In some embodiments of any of the methods described herein, the lymphodepletion agent comprises cyclophosphamide and/or fludarabine.

In some embodiments of any of the methods described herein, the administering comprises initially administering a single dose of the T cells (including T reg cells), however two or more doses of the T cells may be administered depending on the autoimmune disease being treated and its severity. In some embodiments of any of the methods described herein, the administering may comprise administering five or more doses of the T cells. In some embodiments of any of the methods described herein, the administering comprises administering ten or more doses of the T cells.

In some embodiments of any of the methods described herein, the method further comprises, prior to the administering step, a step of generating T reg cells. In some embodiments of any of the methods described herein, the step of generating the T reg cells comprises introducing into a T cell a nucleic acid construct comprising a first sequence encoding the humanized chimeric antigen receptor. In some embodiments of any of the methods described herein, the nucleic acid construct further comprises a promoter operably linked to the sequence encoding the humanized chimeric antigen receptor.

In some embodiments of any of the methods described herein, the step of generating the T reg cells further comprises introducing into the T cell a second sequence encoding a FOXP3 polypeptide. In some embodiments of any of the methods described herein, the nucleic acid construct comprises the second sequence encoding a FOXP3 polypeptide. In some embodiments of any of the methods described herein, the first sequence is positioned 5′ relative the second sequence in the nucleic acid construct. In some embodiments of any of the methods described herein, the nucleic acid construct further comprises an additional sequence between the first sequence and the second sequence, wherein the additional sequence operably links the second sequence to the first sequence.

In some embodiments of any of the methods described herein, the second sequence is 5′ positioned relative to the first sequence in the nucleic acid construct. In some embodiments of any of the methods described herein, the nucleic acid construct further comprises an additional sequence between the second sequence and the first sequence, wherein the additional sequence operably links the first sequence to the second sequence.

In some embodiments of any of the methods described herein, the additional sequence comprises an internal ribosome entry site (IRES) sequence or encodes a self-cleaving amino acid. In some embodiments of any of the methods described herein, the additional sequence comprises one or both of a promoter and an enhancer.

In some embodiments of any of the methods described herein, the nucleic acid construct comprises a viral vector selected from the group consisting of a lentiviral vector, a retroviral vector, an adenoviral vector, transposons, cosmids, and an adeno-associated viral (AAV) vector. In some embodiments of any of the methods described herein, the viral vector is a lentiviral vector. In some embodiments of any of the methods described herein, the introducing comprises viral transduction.

In some embodiments of any of the methods described herein, the step of generating the T reg cells further comprises, prior to the step of introducing the nucleic acid construct into the T cell, a step of obtaining the T cell from the subject or obtaining the T cell from an allogeneic subject. In some embodiments of any of the methods described herein, the step of generating the T reg cells further comprises contacting the T cell with an effective amount of one or more CD3-stimulation agents in the absence of a CD28 stimulating agent for a first period of time under conditions that allow for the stimulation of the T cell.

In some embodiments of any of the methods described herein, the T reg cells are administered using intravenous administration. In some embodiments of any of the methods described herein, the administering results in amelioration of one or more symptoms of the autoimmune disease in the subject.

Also provided herein are methods of making a regulator T cell that include introducing into a T cell a nucleic acid construct comprising a first sequence encoding a humanized chimeric antigen receptor. In some embodiments of any of the methods described herein, the humanized chimeric antigen receptor comprises an extracellular antigen-binding domain, a transmembrane domain, and an intracellular domain, wherein the intracellular domain comprises a cytoplasmic signaling domain and one or more co-stimulatory domains. In some embodiments of any of the methods described herein, the cytoplasmic signaling domain is a CD3 zeta intracellular signaling domain and the co-stimulatory domain comprises an intracellular signaling domain from at least one of 4-1BB, OX40, or CD28. In some embodiments of any of the methods described herein, the extracellular antigen-binding domain is an antibody or an antigen-binding fragment. In some embodiments of any of the methods described herein, the antigen-binding fragment is selected from the group consisting of a Fab, a F(ab′) 2 fragment, a scFv, a scAb, a dAb, and a single domain antibody. In some embodiments of any of the methods described herein, the extracellular antigen-binding domain comprises an scFv that is capable of binding to an antigen on an autoimmune cell. In some embodiments of any of the methods described herein, the antigen is CD19.

In some embodiments of any of the methods described herein, the extracellular antigen-binding domain comprises: (i) a heavy chain variable domain comprising a CDR1, a CDR2, and a CDR3 of SEQ ID NOs: 1-3, respectively; and (ii) a light chain variable domain comprising a CDR1, a CDR2, and a CDR3 of SEQ ID NOs: 4-6, respectively. In some embodiments of any of the methods described herein, the antigen-binding fragment comprises: (i) a heavy chain variable domain that is at least 90% identical to SEQ ID NO: 7; and (ii) a light chain variable domain that is at least 90% identical to SEQ ID NO: 8.

In some embodiments of any of the methods described herein, the humanized chimeric antigen receptor comprises a sequence that is at least 90% identical to SEQ ID NO: 14. In some embodiments of any of the methods described herein, the humanized chimeric antigen receptor comprises a sequence that is at least 95% identical to SEQ ID NO: 14. In some embodiments of any of the methods described herein, the humanized chimeric antigen receptor comprises SEQ ID NO: 14.

In some embodiments of any of the methods described herein, the nucleic acid construct further comprises a promoter operably linked to the sequence encoding the humanized chimeric antigen receptor. In some embodiments of any of the methods described herein, the method further comprises introducing into the T cell a second sequence encoding a FOXP3 polypeptide. In some embodiments of any of the methods described herein, the nucleic acid construct comprises the second sequence encoding a FOXP3 polypeptide. In some embodiments of any of the methods described herein, the first sequence is positioned 5′ relative the second sequence in the nucleic acid construct. In some embodiments of any of the methods described herein, the nucleic acid construct further comprises an additional sequence between the first sequence and the second sequence, wherein the additional sequence operably links the second sequence to the first sequence.

In some embodiments of any of the methods described herein, the second sequence is 5′ positioned relative to the first sequence in the nucleic acid construct. In some embodiments of any of the methods described herein, the nucleic acid construct further comprises an additional sequence between the second sequence and the first sequence, wherein the additional sequence operably links the first sequence to the second sequence.

In some embodiments of any of the methods described herein, the additional sequence comprises an internal ribosome entry site (IRES) sequence or encodes a self-cleaving amino acid. In some embodiments of any of the methods described herein, the additional sequence comprises one or both of a promoter and an enhancer.

In some embodiments of any of the methods described herein, the nucleic acid construct comprises a viral vector selected from the group consisting of a lentiviral vector, a retroviral vector, an adenoviral vector, transposons, cosmids, and an adeno-associated viral (AAV) vector. In some embodiments of any of the methods described herein, the viral vector is a lentiviral vector. In some embodiments of any of the methods described herein, the introducing comprises viral transduction.

In some embodiments of any of the methods described herein, the method further comprises, prior to the step of introducing the nucleic acid construct into the T cell, a step of obtaining the T cell from the subject or obtaining the T cell from an allogeneic subject. In some embodiments of any of the methods described herein, the method further comprises a step of contacting the T cell with an effective amount of one or more CD3-stimulation agents in the absence of a CD28 stimulating agent for a first period of time under conditions that allow for the stimulation of the T cell.

Also provided herein are T reg cells produced by any of the methods described herein. Also provided herein are compositions including any of the T reg cells produced by any of the methods described herein.

Also provided herein are T reg cells comprising a nucleic acid construct comprising a first sequence encoding a humanized chimeric antigen receptor. In some embodiments of any of the T reg cells described herein, the humanized chimeric antigen receptor comprises an extracellular antigen-binding domain, a transmembrane domain, and an intracellular domain, wherein the intracellular domain comprises a cytoplasmic signaling domain and one or more co-stimulatory domains. In some embodiments of any of the T reg cells described herein, the cytoplasmic signaling domain is a CD3 zeta intracellular signaling domain and the co-stimulatory domain comprises an intracellular signaling domain from at least one of 4-1BB, OX40, or CD28. In some embodiments of any of the T reg cells described herein, the extracellular antigen-binding domain is an antibody or an antigen-binding fragment. In some embodiments of any of the T reg cells described herein, the antigen-binding fragment is selected from the group consisting of a Fab, a F(ab′)₂ fragment, a scFv, a scAb, a dAb, and a single domain antibody. In some embodiments of any of the T reg cells described herein, the extracellular antigen-binding domain comprises an scFv that is capable of binding to an antigen on an autoimmune cell. In some embodiments of any of the T reg cells described herein, the antigen is CD19.

In some embodiments of any of the T reg cells described herein, the extracellular antigen-binding domain comprises: (i) a heavy chain variable domain comprising a CDR1, a CDR2, and a CDR3 of SEQ ID NOs: 1-3, respectively; and (ii) a light chain variable domain comprising a CDR1, a CDR2, and a CDR3 of SEQ ID NOs: 4-6, respectively. In some embodiments of any of the T reg cells described herein, the antigen-binding fragment comprises: (i) a heavy chain variable domain that is at least 90% identical to SEQ ID NO: 7; and (ii) a light chain variable domain that is at least 90% identical to SEQ ID NO: 8. In some embodiments of any of the T reg cells described herein, the humanized chimeric antigen receptor comprises a sequence that is at least 90% identical to SEQ ID NO: 14. In some embodiments of any of the T reg cells described herein, the humanized chimeric antigen receptor comprises a sequence that is at least 95% identical to SEQ ID NO: 14. In some embodiments of any of the T reg cells described herein, the humanized chimeric antigen receptor comprises SEQ ID NO: 14.

In some embodiments of any of the T reg cells described herein, the nucleic acid construct further comprises a promoter operably linked to the sequence encoding the humanized chimeric antigen receptor. In some embodiments of any of the T reg cells described herein, the T reg cell further comprises a second sequence encoding a FOXP3 polypeptide.

In some embodiments of any of the T reg cells described herein, the nucleic acid construct comprises the second sequence encoding a FOXP3 polypeptide. In some embodiments of any of the T reg cells described herein, the first sequence is positioned 5′ relative the second sequence in the nucleic acid construct. In some embodiments of any of the T reg cells described herein, the nucleic acid construct further comprises an additional sequence between the first sequence and the second sequence, wherein the additional sequence operably links the second sequence to the first sequence.

In some embodiments of any of the T reg cells described herein, the second sequence is 5′ positioned relative to the first sequence in the nucleic acid construct. In some embodiments of any of the T reg cells described herein, the nucleic acid construct further comprises an additional sequence between the second sequence and the first sequence, wherein the additional sequence operably links the first sequence to the second sequence.

In some embodiments of any of the T reg cells described herein, the additional sequence comprises an internal ribosome entry site (IRES) sequence or encodes a self-cleaving amino acid. In some embodiments of any of the T reg cells described herein, the additional sequence comprises one or both of a promoter and an enhancer.

In some embodiments of any of the T reg cells described herein, the nucleic acid construct comprises a viral vector selected from the group consisting of a lentiviral vector, a retroviral vector, an adenoviral vector, transposons, cosmids, and an adeno-associated viral (AAV) vector. In some embodiments of any of the T reg cells described herein, the viral vector is a lentiviral vector.

Also provided herein are compositions that include any of the T reg cells described herein.

Also provided herein are nucleic acid constructs that comprise a first sequence encoding a humanized chimeric antigen receptor and a second sequence encoding a FOXP3 polypeptide. In some embodiments of any of the nucleic acid constructs described herein, the humanized chimeric antigen receptor comprises an extracellular antigen-binding domain, a transmembrane domain, and an intracellular domain, wherein the intracellular domain comprises a cytoplasmic signaling domain and one or more co-stimulatory domains. In some embodiments of any of the nucleic acid constructs described herein, the cytoplasmic signaling domain is a CD3 zeta intracellular signaling domain and the co-stimulatory domain comprises an intracellular signaling domain from at least one of 4-1BB, OX40, or CD28. In some embodiments of any of the nucleic acid constructs described herein, the extracellular antigen-binding domain is an antibody or an antigen-binding fragment. In some embodiments of any of the nucleic acid constructs described herein, the antigen-binding fragment is selected from the group consisting of a Fab, a F(ab′) 2 fragment, a scFv, a scAb, a dAb, and a single domain antibody. In some embodiments of any of the nucleic acid constructs described herein, the extracellular antigen-binding domain comprises an scFv that is capable of binding to an antigen on an autoimmune cell. In some embodiments of any of the nucleic acid constructs described herein, the antigen is CD19.

In some embodiments of any of the nucleic acid constructs described herein, the extracellular antigen-binding domain comprises: (i) a heavy chain variable domain comprising a CDR1, a CDR2, and a CDR3 of SEQ ID NOs: 1-3, respectively; and (ii) a light chain variable domain comprising a CDR1, a CDR2, and a CDR3 of SEQ ID NOs: 4-6, respectively. In some embodiments of any of the nucleic acid constructs described herein, the antigen-binding fragment comprises: (i) a heavy chain variable domain that is at least 90% identical to SEQ ID NO: 7; and (ii) a light chain variable domain that is at least 90% identical to SEQ ID NO: 8.

In some embodiments of any of the nucleic acid constructs described herein, the humanized chimeric antigen receptor comprises a sequence that is at least 90% identical to SEQ ID NO: 14. In some embodiments of any of the nucleic acid constructs described herein, the humanized chimeric antigen receptor comprises a sequence that is at least 95% identical to SEQ ID NO: 14. In some embodiments of any of the nucleic acid constructs described herein, the humanized chimeric antigen receptor comprises SEQ ID NO: 14.

In some embodiments of any of the nucleic acid constructs described herein, the nucleic acid construct further comprises a promoter operably linked to the sequence encoding the humanized chimeric antigen receptor. In some embodiments of any of the nucleic acid constructs described herein, the first sequence is positioned 5′ relative the second sequence in the nucleic acid construct. In some embodiments of any of the nucleic acid constructs described herein, the nucleic acid construct further comprises an additional sequence between the first sequence and the second sequence, wherein the additional sequence operably links the second sequence to the first sequence.

In some embodiments of any of the nucleic acid constructs described herein, the second sequence is 5′ positioned relative to the first sequence in the nucleic acid construct. In some embodiments of any of the nucleic acid constructs described herein, the nucleic acid construct further comprises an additional sequence between the second sequence and the first sequence, wherein the additional sequence operably links the first sequence to the second sequence. In some embodiments of any of the nucleic acid constructs described herein, the additional sequence comprises an internal ribosome entry site (IRES) sequence or encodes a self-cleaving amino acid. In some embodiments of any of the nucleic acid constructs described herein, the additional sequence comprises one or both of a promoter and an enhancer.

In some embodiments of any of the nucleic acid constructs described herein, the nucleic acid construct comprises a viral vector selected from the group consisting of a lentiviral vector, a retroviral vector, transposons, cosmids, an adenoviral vector, and an adeno-associated viral (AAV) vector. In some embodiments of any of the nucleic acid constructs described herein, the viral vector is a lentiviral vector.

Also provided herein are compositions that include any of the nucleic acid constructs described herein.

Also provided herein are kits that include any of the compositions described herein.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 are graphs showing cytotoxic activity of Systemic Lupus Erythematosus (SLE) patient-derived peripheral blood mononuclear cells (PBMCs) transduced with a Hu19-CD828Z chimeric antigen receptor (CAR) construct against NALM6 (CD19⁺) cells.

FIG. 2 are graphs showing cytotoxic activity of SLE patient- and Healthy Donor Derived-PBMCs transduced with a Hu19-CD828Z CAR construct against autologous primary B cells expressing CD19.

FIGS. 3A-B are graphs showing interferon-gamma (IFN□) release by SLE patient-derived PBMCs transduced with a Hu19-CD828Z CAR construct following co-culture with NALM6 (CD19⁺) tumor cells (FIG. 3A) or with autologous primary B cells expressing CD19 (FIG. 3B).

FIGS. 4A-C are graphs showing Hu19-CD828Z transduced PBMC proliferation following co-culture with NALM6 (CD19⁺) tumor cells (FIG. 4A), autologous primary B cells expressing CD19 (FIG. 4B), or control K562 (CD19⁻) cells (FIG. 4C).

### DETAILED DESCRIPTION

B cells express a wide array of cell surface molecules during their differentiation and proliferation including, e.g., surface antigens CD19 and CD20.

The present disclosure provides methods and materials that can be used to treat a subject identified as having an autoimmune disease. For example, this document provides materials and methods for treating B cell associated autoimmune diseases. Examples of B cell associated autoimmune diseases include, but are not limited to, dermatomyositis, myasthenia gravis, systemic lupus erythematosus, lupus nephritis, Addison's disease, rheumatoid arthritis, multiple sclerosis, Goodpasture's syndrome, Sjogren's syndrome, thyrotoxicosis, scleroderma, chronic active hepatitis, polymyositis/dermatomyositis, polychondritis, pamphigus vulgaris, and amyotrophic lateral sclerosis.

Thus, provided herein are methods of reducing the number of B cells in a tissue in a subject having an autoimmune disease, methods of treating a subject having an autoimmune disease, T cells, and nucleic acid constructs.

### Chimeric Antigen Receptors

As used herein, the term “chimeric antigen receptor” or “CAR” refers to a chimeric antigen receptor comprising an extracellular domain, a transmembrane domain, and an intracellular domain. In some cases, the extracellular domain can comprise an antigen-binding domain. In some cases, the transmembrane domain can comprise a transmembrane domain derived from a natural polypeptide obtained from a membrane-binding or transmembrane protein. For example, a transmembrane domain can include, without limitation, a transmembrane domain from a T cell receptor alpha or beta chain, a CD3 zeta chain, a CD28 polypeptide, or a CD8 polypeptide. In some cases, the intracellular domain can comprise a cytoplasmic signaling domain (e.g., any of the cytoplasmic signaling domains described herein) and one or more co-stimulatory domains (e.g., any of the exemplary co-stimulatory domains described herein).

In some embodiments of any of the chimeric antigen receptors described herein, the chimeric antigen receptor can be a humanized chimeric antigen receptor comprising an extracellular antigen-binding domain, a transmembrane domain, and an intracellular domain, wherein the intracellular domain comprises a cytoplasmic signaling domain and one or more co-stimulatory domains.

As used herein, the term “stimulation” refers to stage of CAR signaling where a co-stimulatory signal can be used to achieve a robust and sustained CAR signaling response.

In some embodiments, a co-stimulatory domain can be an intracellular signaling domain from a polypeptide selected from the group consisting of: CD27, CD28, OX40, CD30, CD40, B7-H3, NKG2C, LIGHT, CD7, CD2, 4-1BB, and PD-1.

In some embodiments, the extracellular domain of the chimeric antigen receptor can comprise a hinge region. In some embodiments, the hinge region can comprise an extracellular hinge region from a CD28 polypeptide or a fragment thereof. The hinge region is a short sequence of amino acids that can facilitate flexibility of the extracellular antigen-binding domain (see, e.g. Woof et al., Nat. Rev. Immunol. 4 (2): 89-99 (2004)), and can be positioned between the extracellular antigen-binding domain and the transmembrane domain. The hinge region can include all, or a portion of, an extracellular region of any transmembrane protein. In some embodiments, the hinge region is derived from the human CD8α protein or the human CD28 protein.

In some embodiments, the extracellular antigen-binding domain is an antibody or an antigen-binding fragment. Additional non-limiting examples and aspects of extracellular antigen-binding domains are described below.

The transmembrane domain of a chimeric antigen receptor can be any transmembrane domain derived or obtained from any protein known in the art. For example, the transmembrane domain can be obtained or derived from a CD8α protein (e.g., a human CD8α protein) or a CD28 protein (e.g., a human CD28 protein). CD8 is a transmembrane glycoprotein that functions as a co-receptor for the T-cell receptor (TCR), and is expressed primarily on the surface of cytotoxic T-cells. The most common form of CD8 exists as a dimer composed of a CD8α and CD8β chain. CD28 is expressed on T-cells and provides co-stimulatory signals required for T-cell activation. CD28 is the receptor for CD80 (B7.1) and CD86 (B7.2). In some embodiments, where the chimeric antigen receptor polypeptide includes a CD28 transmembrane domain, the CD28 transmembrane domain is at least 80% (e.g., at least 85% 90%, 95%, 99%, or 100%) identical to: FWVLVVVGGVLACYSLLVTVAFIIFWV (SEQ ID NO: 12).

The most common cytoplasmic signaling domain included in chimeric antigen receptors is an intracellular signaling domain of CD3 zeta (CD32). CD3 zeta associates with T cell receptors to produce a signal and contains immuno-receptor tyrosine-based activation motifs (ITAMs). In some embodiments, where the cytoplasmic signaling domain is a CD3 zeta intracellular signaling domain, the CD3 zeta intracellular signaling domain has an amino acid sequence that is at least 80% (e.g., at least 85%, 90%, 95%, 99%, or 100%) identical to:
MKWKALFTAAILQAQLPITEAQSFGLLDPKLCYLLDGILF IYGVILTALF LRVKFSRSADAPAYQQGQNQ LYNELNLGRR EEYDVLDKRR GRDPEMGGKP QRRKNPQEGL YNELQKDKMAEAYSEIGMKG ERRRGKGHDG LYQGLSTATK DTYDALHMQA LPPR (NCBI Reference No.: NP_932170) (SEQ ID NO: 10), or a fragment thereof that has activating or stimulatory activity.

In some embodiments of any of the chimeric antigen receptors described herein, the chimeric antigen receptor comprises one or more (e.g., two, three, four, or five) co-stimulatory domains. In some embodiments, the one or more co-stimulatory domains can comprise an intracellular signaling domain from a polypeptide selected from the group of: 4-1BB, OX40, CD28, CD27, and the gamma chain of a human high-affinity IgE receptor (FcγRI). CD28 is a well-known T cell marker important in T cell co-stimulation. 4-1BB (also known as CD137) can transmit a potent costimulatory signal to T cells that induces differentiation and enhances long-term survival of T lymphocytes. CD27 is a member of the TNF receptor superfamily, and is required for generation and long-term maintenance of T-cell immunity. The human high-affinity IgE receptor (FcεRI) is a tetrameric receptor complex consisting of one alpha, one beta, and two disulfide bridge connected gamma chains. FcεRI is constitutively expressed on mast cells and basophils and is inducible in eosinophils. In some embodiments, the intracellular T-cell signaling domains are human.

In some embodiments, where the chimeric antigen receptor comprises a co-stimulatory domain that comprises an intracellular signaling domain from CD28. In some embodiments, the co-stimulatory domain comprises a sequence that is at least 80% (e.g., at least 85%, 90%, 95%, 99%, or 100%) identical to:

(SEQ ID NO: 11)
IEVMYPPPYLDNEKSNGTIIHVKGKHLCPSPLFPGPSKPF
WVLVVVGGVLACYSLLVTVAFIIFWVRSKRSRLLHSDYMN
MTPRRPGPTRKHYQPYAPPRDFAAY.

In some embodiments of any of the chimeric antigen receptors described herein, the cytoplasmic signaling domain is a CD3 zeta intracellular signaling domain and the co-stimulatory domain comprises an intracellular signaling domain from at least one of 4-1BB, OX40, or CD28.

In some embodiments, the chimeric antigen receptor can include a CD28 transmembrane domain, a cytoplasmic signaling domain that is an intracellular signaling domain of CD35, and a co-stimulatory domain that is an intracellular signaling domain of CD28.

In some embodiments, the chimeric antigen receptor can include a CD8α transmembrane domain, a cytoplasmic signaling domain that is an intracellular signaling domain of CD35, and one or more co-stimulatory domains that are intracellular signaling domains of a polypeptide selected from the group consisting of: CD28, the gamma chain of FcεRI, and/or 4-1BB.

In some embodiments, the chimeric antigen receptor includes a CD8α transmembrane domain, a cytoplasmic signaling domain that is an intracellular signaling domain of CD35, and one or more co-stimulatory domains that are intracellular signaling domains of a polypeptide selected from the group consisting of CD28 and CD27.

In some embodiments, the chimeric antigen receptor includes a CD28 transmembrane domain, a cytoplasmic signaling domain that is an intracellular signaling domain of CD35, and one or more co-stimulatory domains that are intracellular signaling domains of a polypeptide selected from the group consisting of: CD27, 4-1BB, and the gamma chain of FcεRI.

The present disclosure also includes functional variants of any of the chimeric antigen receptors described herein. The term “functional variant,” as used herein, refers to a chimeric antigen receptor, a polypeptide, or a protein having substantial sequence identity or similarity to the chimeric antigen receptor, where the functional variant retains the biological activity or function of the chimeric antigen receptor. Functional variants encompass, for example, variants of the chimeric antigen receptors described herein (the parent chimeric antigen receptor) that retain the ability to recognize target cells to a similar extent, the same extent, or to a higher extent, as the parent chimeric antigen receptor. In reference to a nucleic acid sequence encoding the parent chimeric antigen receptor, a nucleic acid sequence encoding a functional variant of the chimeric antigen receptor can be for example, about 10% identical, about 25% identical, about 30% identical, about 50% identical, about 65% identical, about 80% identical, about 90% identical, about 95% identical, or about 99% identical to the nucleic acid sequence encoding the parent chimeric antigen receptor. For example, the parent chimeric antigen receptor includes, without limitation, a chimeric antigen receptor comprising the sequence of SEQ ID NO: 14.

Alternatively or additionally, functional variants can include amino acid sequences of the parent chimeric antigen receptor with at least one non-conservative amino acid substitution. A “non-conservative mutation” involves amino acid substitutions between different amino acid groups, for example, lysine for tryptophan, or phenylalanine for serine, etc. In some embodiments, the non-conservative amino acid substitution(s) do not compromise or inhibit the biological activity of, the functional variant. The non-conservative amino acid substitution may enhance the biological activity of the functional variant, such that the biological activity of the functional variant is increased relative to the parent chimeric antigen receptor.

The present disclosure also includes chimeric antigen receptors directed to any target molecule of interest that includes any of the extracellular antigen-binding domains, transmembrane domains, any of the cytoplasmic signaling domains, and one or more of any of the co-stimulatory domains described herein, in any combination.

For example, a chimeric antigen receptor can comprise (i) a hinge region, (ii) a transmembrane domain derived from human CD8α, (iii) a cytoplasmic signaling domain that is a human CD3ζ intracellular signaling domain; and (iv) a co-stimulatory domain comprising an intracellular signaling domain from human CD28 (as employed in the chimeric antigen receptor of SEQ ID NO: 14).

In some embodiments, the chimeric antigen receptor can comprise (i) a hinge region, (ii) a transmembrane domain derived from human CD8α, (iii) a cytoplasmic signaling domain that is a human CD32 intracellular signaling domain; and (iv) a co-stimulatory domain comprising an intracellular signaling domain from a protein selected from the group consisting of: human CD28 and human CD27.

In some embodiments, the chimeric antigen receptor can comprise (i) a hinge region, (ii) a transmembrane domain derived from human CD8α, (iii) a cytoplasmic signaling domain that is a human CD3ζ intracellular signaling domain; and (iv) a co-stimulatory domain comprising an intracellular signaling domain from a protein selected from the group consisting of: human CD28, human CD27, and the gamma chain of human FcεRI.

In some embodiments, the chimeric antigen receptor can comprise (i) a hinge region, (ii) a transmembrane domain derived from human CD8α, (iii) a cytoplasmic signaling domain that is a human CD3 intracellular signaling domain; and (iv) a co-stimulatory domain comprising an intracellular signaling domain from a protein selected from the group consisting of: human CD28 and the gamma chain of human FcεRI.

In some embodiments of any of the chimeric antigen receptors described herein, the chimeric antigen receptor can further include a signal sequence. The signal sequence may be positioned at the amino terminus of the extracellular antigen-binding domain. The signal sequence can include any suitable signal sequence. In some embodiments, the signal sequence is a human granulocytemacrophage colony-stimulating factor (GM-CSF) receptor signal sequence or a CD8α signal sequence. For example, a chimeric antigen receptor comprising a humanized scFv can comprise a CD8α signal sequence.

In some embodiments, a chimeric antigen receptor (e.g., a humanized chimeric antigen receptor) comprises a sequence that is at least 80%, at least 85%, at least 90%, at least 92%, at least 94%, at least 96%, at least 98%, at least 99%, or at least 100% identical to SEQ ID NO: 14.

### Antigen-Binding Domains

As used herein, the term “antigen-binding moiety” refers to an intact immunoglobulin or to an antigen-binding fragment thereof. Antigen-binding fragments may be produced by recombinant DNA techniques or by enzymatic or chemical cleavage of intact antibodies. Examples of antigen-binding fragments include Fab, Fab′, F(ab′)₂, Fv, domain antibodies (dAbs), single-chain antibodies (scFv), chimeric antibodies, diabodies, triabodies, tetrabodies, scAbs, single domain antibodies (e.g., VHH or VNAR), and polypeptides that contain at least a portion of an immunoglobulin that is sufficient to confer specific antigen-binding to the polypeptide.

As used herein, the term “scFv” comprises the V_{H} and V_{L} domains of an antibody, wherein these domains are present in a single polypeptide chain.

In some embodiments, the extracellular antigen-binding domain is human or humanized.

In some embodiments, any of the antigen-binding domain can bind specifically to a target. For example, the antigen-binding domain can bind specifically to CD19 (e.g., an anti-CD19 scFv).

As used herein, cluster of differentiation 19 (e.g., “CD19”) encodes a member of the immunoglobulin gene superfamily, however, expression of this cell surface protein is restricted to B cell lymphocytes. CD19 has two N-terminal extracellular Ig-like domains separated by a non-Ig-like domain, a hydrophobic transmembrane domain, and a large C-terminal cytoplasmic domain. This protein forms a complex with several membrane proteins including complement receptor type 2 (CD21) and tetraspanin (CD81) and this complex reduces the threshold for antigen-initiated B cell activation. Activation of this B-cell antigen receptor complex activates the phosphatidylinositol 3-kinase signaling pathway and the subsequent release of intracellular stores of calcium ions. An example of a human CD19 polypeptide includes, without limitation, NCBI reference sequence: NP_001171569.1, or a fragment thereof.

In some embodiments, an extracellular antigen-binding domain can comprise a variable region of an anti-CD19 monoclonal antibody. An anti-CD19 monoclonal antibody can be obtained or derived from a subject, including but not limited to, a mouse, a rat, or a human. The extracellular antigen-binding domain can comprises a variable region of a mouse or human anti-CD19 monoclonal antibody. In some embodiments, the extracellular antigen-binding domain includes a light chain variable region and a heavy chain variable region of a mouse, human, or humanized anti-CD19 monoclonal antibody. The 47G4 antibody (described in U.S. Patent Application Publication No. 2010/0104509, which is incorporated herein by reference in its entirety) is one example of a human anti-CD19 monoclonal antibody that can be used in the present disclosure.

In some embodiments, the extracellular antigen-binding domain comprises: (i) a heavy chain variable domain comprising a CDR1, a CDR2, and a CDR3 of SEQ ID NOs: 1-3, respectively; and (ii) a light chain variable domain comprising a CDR1, a CDR2, and a CDR3 of SEQ ID NOs: 4-6, respectively. In some embodiments, the extracellular antigen-binding domain comprises: (i) a heavy chain variable domain that is at least 90% identical to SEQ ID NO: 7; and (ii) a light chain variable domain that is at least 90% identical to SEQ ID NO: 8. In some embodiments, the extracellular antigen-binding domain comprises: (i) a heavy chain variable domain that is at least 95% identical to SEQ ID NO: 7; and (ii) a light chain variable domain that is at least 95% identical to SEQ ID NO: 8. In some embodiments, the extracellular antigen-binding domain comprises: (i) a heavy chain variable domain comprises SEQ ID NO: 7; and (ii) a light chain variable domain comprises SEQ ID NO: 8.

As used herein, “FOXP3” refers to the FOXP3 gene or protein that is a transcription factor in the Forkhead box (Fox) family of transcription factors (Sakaguchi et al., Int'l Immun., 21 (10): 1105-1111 (2009); Pandiyan, et al., Cytokine, 76 (1): 13-24 (2015)), or a variant thereof (e.g., a FOXP3 protein having one or more (e.g., one, two, three, four, five, six, seven, eight, nine, ten, eleven, twelve, thirteen, fourteen, fifteen, sixteen, seventeen, eighteen, nineteen, or twenty amino acid substitutions, amino acid deletions, or amino acid insertions as compared to a wildtype FOXP3 protein). In some embodiments, when preparing a T reg cell to be used in the treatment of a subject having an autoimmune disease, FOXP3 refers to human FOXP3 or a variant thereof. An example of a wildtype human FOXP3 polypeptide includes, without limitation, NCBI reference sequence: NP 001107849.1 or a fragment thereof.

In some embodiments, a human FOXP3 polypeptide comprises a sequence that is at least 80% identical (e.g., at least 85%, at least 90%, at least 92%, at least 94%, at least 96%, at least 98%, at least 99%, or 100% identical) to:

(SEQ ID NO: 15)
MPNPRPGKPSAPSLALGPSPGASPSWRAAPKASDL
LGARGPGGTFQGRDLRGGAHASSSSLNPMPPSQLQ
LSTVDAHARTPVLQVHPLESPAMISLTPPTTATGV
FSLKARPGLPPGINVASLEWVSREPALLCTFPNPS
APRKDSTLSAVPQSSYPLLANGVCKWPGCEKVFEE
PEDFLKHCQADHLLDEKGRAQCLLQREMVQSLEQQ
LVLEKEKLSAMQAHLAGKMALTKASSVASSDKGSC
CIVAAGSQGPVVPAWSGPREAPDSLFAVRRHLWGS
HGNSTFPEFLHNMDYFKFHNMRPPFTYATLIRWAI
LEAPEKQRTLNEIYHWFTRMFAFFRNHPATWKNAI
RHNLSLHKCFVRVESEKGAVWTVDELEFRKKRSQR
PSRCSNPTPGP

In some embodiments, a human FOXP3 polypeptide can be encoded by a nucleic acid that is at least 80% identical (e.g., at least 85%, at least 90%, at least 92%, at least 94%, at least 96%, at least 98%, at least 99%, or 100% identical) to:

(SEQ ID NO: 13)
AGTTTCCCACAAGCCAGGCTGATCCTTTTCTGTCA
GTCCACTTCACCAAGCCTGCCCTTGGACAAGGACC
CGATGCCCAACCCCAGGCCTGGCAAGCCCTCGGCC
CCTTCCTTGGCCCTTGGCCCATCCCCAGGAGCCTC
GCCCAGCTGGAGGGCTGCACCCAAAGCCTCAGACC
TGCTGGGGGCCCGGGGCCCAGGGGGAACCTTCCAG
GGCCGAGATCTTCGAGGCGGGGCCCATGCCTCCTC
TTCTTCCTTGAACCCCATGCCACCATCGCAGCTGC
AGCTCTCAACGGTGGATGCCCACGCCCGGACCCCT
GTGCTGCAGGTGCACCCCCTGGAGAGCCCAGCCAT
GATCAGCCTCACACCACCCACCACCGCCACTGGGG
TCTTCTCCCTCAAGGCCCGGCCTGGCCTCCCACCT
GGGATCAACGTGGCCAGCCTGGAATGGGTGTCCAG
GGAGCCGGCACTGCTCTGCACCTTCCCAAATCCCA
GTGCACCCAGGAAGGACAGCACCCTTTCGGCTGTG
CCCCAGAGCTCCTACCCACTGCTGGCAAATGGTGT
CTGCAAGTGGCCCGGATGTGAGAAGGTCTTCGAAG
AGCCAGAGGACTTCCTCAAGCACTGCCAGGCGGAC
CATCTTCTGGATGAGAAGGGCAGGGCACAATGTCT
CCTCCAGAGAGAGATGGTACAGTCTCTGGAGCAGC
AGCTGGTGCTGGAGAAGGAGAAGCTGAGTGCCATG
CAGGCCCACCTGGCTGGGAAAATGGCACTGACCAA
GGCTTCATCTGTGGCATCATCCGACAAGGGCTCCT
GCTGCATCGTAGCTGCTGGCAGCCAAGGCCCTGTC
GTCCCAGCCTGGTCTGGCCCCCGGGAGGCCCCTGA
CAGCCTGTTTGCTGTCCGGAGGCACCTGTGGGGTA
GCCATGGAAACAGCACATTCCCAGAGTTCCTCCAC
AACATGGACTACTTCAAGTTCCACAACATGCGACC
CCCTTTCACCTACGCCACGCTCATCCGCTGGGCCA
TCCTGGAGGCTCCAGAGAAGCAGCGGACACTCAAT
GAGATCTACCACTGGTTCACACGCATGTTTGCCTT
CTTCAGAAACCATCCTGCCACCTGGAAGAACGCCA
TCCGCCACAACCTGAGTCTGCACAAGTGCTTTGTG
CGGGTGGAGAGCGAGAAGGGGGCTGTGTGGACCGT
GGATGAGCTGGAGTTCCGCAAGAAACGGAGCCAGA
GGCCCAGCAGGTGTTCCAACCCTACACCTGGCCCC
TGACCTCAAGATCAAGGAAAGGAGGATGGACGAAC
AGGGGCCAAACTGGTGGGAGGCAGAGGTGGTGGGG
GCAGGGATGATAGGCCCTGGATGTGCCCACAGGGA
CCAAGAAGTGAGGTTTCCACTGTCTTGCCTGCCAG
GGCCCCTGTTCCCCCGCTGGCAGCCACCCCCTCCC
CCATCATATCCTTTGCCCCAAGGCTGCTCAGAGGG
GCCCCGGTCCTGGCCCCAGCCCCCACCTCCGCCCC
AGACACACCCCCCAGTCGAGCCCTGCAGCCAAACA
GAGCCTTCACAACCAGCCACACAGAGCCTGCCTCA
GCTGCTCGCACAGATTACTTCAGGGCTGGAAAAGT
CACACAGACACACAAAATGTCACAATCCTGTCCCT
CACTCAACACAAACCCCAAAACACAGAGAGCCTGC
CTCAGTACACTCAAACAACCTCAAAGCTGCATCAT
CACACAATCACACACAAGCACAGCCCTGACAACCC
ACACACCCCAAGGCACGCACCCACAGCCAGCCTCA
GGGCCCACAGGGGCACTGTCAACACAGGGGTGTGC
CCAGAGGCCTACACAGAAGCAGCGTCAGTACCCTC
AGGATCTGAGGTCCCAACACGTGCTCGCTCACACA
CACGGCCTGTTAGAATTCACCTGTGTATCTCACGC
ATATGCACACGCACAGCCCCCCAGTGGGTCTCTTG
AGTCCCGTGCAGACACACACAGCCACACACACTGC
CTTGCCAAAAATACCCCGTGTCTCCCCTGCCACTC
ACCTCACTCCCATTCCCTGAGCCCTGATCCATGCC
TCAGCTTAGACTGCAGAGGAACTACTCATTTATTT
GGGATCCAAGGCCCCCAACCCACAGTACCGTCCCC
AATAAACTGCAGCCGAGCTCCCCA.

### Nucleic Acid Constructs

Also provided herein are nucleic acid constructs that encode any of the polypeptides described herein. For example, a nucleic acid construct as described herein can include a first sequence that encodes any of the chimeric antigen receptors (e.g., any of the exemplary humanized chimeric antigen receptors described herein). In some embodiments, the nucleic acid construct further comprises a promoter operably linked to the first sequence encoding the chimeric antigen receptor (e.g., humanized chimeric antigen receptor) (e.g., any of the exemplary chimeric antigen receptors described herein).

In some embodiments, the nucleic acid construct can further comprise a second sequence encoding a FOXP3 polypeptide (e.g., a human FOXP3 polypeptide or any of the other exemplary FOXP3 polypeptides described herein). In some embodiments, the first sequence is positioned 5′ related to the second sequence in the nucleic acid construct. In some embodiments, the nucleic acid construct further comprises an additional sequence between the first sequence and the second sequence, wherein the additional sequence operably links the second sequence to the first sequence.

In some embodiments, the second sequence is positioned 5′ relative to the first sequence in the nucleic acid construct. In some embodiments, the nucleic acid construct further comprises an additional sequence between the second sequence and the first sequence, wherein the additional sequence operably links the first sequence to the second sequence.

In some embodiments, the additional sequence comprises an internal ribosome entry site (IRES) sequence or encodes a self-cleaving amino acid. In some embodiments, the additional sequence comprises one or both of a promoter and an enhancer.

Any of the nucleic acid constructs described herein can be an expression vector. For example, an expression vector can include a promoter sequence operably linked to the sequence encoding any of the polypeptides described herein.

Non-limiting examples of nucleic acid constructs include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. In some embodiments, the nucleic acid construct is a viral vector selected from the group consisting of: a lentiviral vector, a retroviral vector, an adenoviral vector, and an adeno-associated viral (AAV) vector.

In some cases, a nucleic acid construct can include sufficient cis-acting elements (e.g., a promoter and/or an enhancer) that supplement expression where the remaining elements needed for expression can be supplied by the host mammalian cell or in an in vitro expression system. Skilled practitioners will be capable of selecting suitable nucleic acid constructs for making any of the T reg cells described herein. Any appropriate promoter (e.g., EF1 alpha) can be operably linked to any of the sequences encoding polypeptides described herein. Non-limiting examples of promoters to be used in any of the nucleic acid constructs described herein include EF1a, SFFV, PGK, CMV, CAG, UbC, MSCV, MND, EF1a hybrid, and/or CAG hybrid promoters.

As used herein, the term “operably linked” is well known in the art and refers to genetic components that are combined such that they carry out their normal functions. For example, a nucleic acid sequence is operably linked to a promoter when its transcription is under the control of the promoter. In another example, a nucleic acid sequence can be operably linked to other nucleic acid sequence by a nucleic acid sequence encoding a self-cleaving 2A polypeptide or a sequence comprising an internal ribosome entry site (IRES).

### Methods of Making T Cells

Also provided herein are methods of making a T reg cell, the method comprising introducing into a T cell a nucleic acid construct comprising a first sequence encoding a humanized chimeric antigen receptor (e.g., any of the exemplary humanized chimeric antigen receptors described herein). In some embodiments, the method further comprises introducing into the T cell a second sequence encoding a FOXP3 polypeptide (e.g., a human FOXP3 polypeptide or any of the other exemplary FOXP3 polypeptides described herein). In some embodiments, the nucleic acid construct further comprises the second sequence encoding a FOXP3 polypeptide. In some embodiments, the first sequence is positioned 5′ relative to the second sequence in the nucleic acid construct. In some embodiments, the nucleic acid construct further comprises an additional sequence between the first sequence and the second sequence, wherein the additional sequence operably links the second sequence to the first sequence.

In some embodiments, the second sequence is positioned 5′ relative to the first sequence in the nucleic acid construct. In some embodiments, the nucleic acid construct further comprises an additional sequence between the second sequence and the first sequence, wherein the additional sequence operably links the first sequence to the second sequence.

In some embodiments, the additional sequence comprises an internal ribosome entry site (IRES) sequence or encodes a self-cleaving amino acid. In some embodiments, the nucleic acid construct comprises one or both of a promoter and an enhancer.

In some embodiments, the nucleic acid construct comprises a viral vector selected from the group consisting of a lentiviral vector, a retroviral vector, an adenoviral vector, transposons, cosmids, and an adeno-associated viral (AAV) vector. In some embodiments, the viral vector is a lentiviral vector. In some embodiments, the step of introducing comprises the use of viral transduction.

Some embodiments of these methods further include, prior to the step of introducing the nucleic acid construct into the T cell, a step of obtaining the T cell from the subject or obtaining the T cell from an allogeneic subject. In some embodiments, a T cell can be isolated from a subject (e.g., a human) using an appropriate method (e.g., magnetic activated cell sorting or flow cytometry-mediated sorting). In some cases,

Some embodiments of any of the methods further include a step of contacting the T cell with an effective amount of one or more CD3-stimulation agents in the absence of a CD28 stimulating agent for a first period of time under conditions that allow for the stimulation of the T cell.

Some embodiments of these methods can include introducing a first nucleic acid construct encoding a chimeric antigen receptor (e.g., a humanized chimeric antigen receptor) (e.g., any of the exemplary chimeric antigen receptors described herein) and a second nucleic acid construct encoding a FOXP3 polypeptide (e.g., a human FOXP3 polypeptide) (e.g., any of the exemplary FOXP3 polypeptides described herein).

Methods of introducing nucleic acid constructs into a cell (e.g., a eukaryotic cell) are known in the art. Non-limiting examples of methods that can be used to introduce a nucleic acid construct into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection. As used herein, “transformed” and “transduced” are used interchangeably.

As used herein a “non-toxic cell” refers to a cell that has been transduced (e.g., by any of the methods described herein) to express a therapeutically effective amount of a chimeric antigen receptor (e.g., any of the chimeric antigen receptors described herein) and administered to a subject, such that the cell retains its biological activity without generating any significant adverse events in the subject. For example, a non-limiting example of an adverse event in a subject is cytokine release syndrome. A non-toxic cell includes any of the cells described herein, including, but not limited to T cells and T reg cells.

### T Reg Cells

Also provided herein are T reg cells produced using any of the methods described herein. T reg cells are thought to be directed to self-antigens that are encountered in the thymus during their development.

Also provided herein are T reg cells comprising a nucleic acid construct (e.g., any of the exemplary nucleic acid constructs described herein) comprising a first sequence encoding a chimeric antigen receptor (e.g., a humanized chimeric antigen receptor) (e.g., any of the exemplary chimeric antigen receptors described herein).

In some embodiments, the nucleic acid construct further comprises a promoter operably linked to the sequence encoding the chimeric antigen receptor (e.g., humanized chimeric antigen receptor).

In some embodiments, the T reg cell further comprises a second sequence encoding a FOXP3 polypeptide (e.g., a human FOXP3 polypeptide) (e.g., any of the exemplary FOXP3 polypeptides described herein). In some embodiments, the nucleic acid construct comprises the second sequence encoding the FOXP3 polypeptide. In some embodiments, the first sequence is positioned 5′ relative to the second sequence in the nucleic acid construct. In some embodiments, the nucleic acid construct further comprises an additional sequence between the first sequence and the second sequence, wherein the additional sequence operably links the second sequence to the first sequence.

In some embodiments, the second sequence is 5′ positioned relative to the first sequence in the nucleic acid construct. In some embodiments, the nucleic acid construct further comprises an additional sequence between the second sequence and the first sequence, wherein the additional sequence operably links the first sequence to the second sequence.

In some embodiments, the additional sequence comprises an internal ribosome entry site (IRES) sequence or encodes a self-cleaving amino acid. In some embodiments, the additional sequence comprises one or both of a promoter and an enhancer.

In some embodiments, the nucleic acid construct comprises a viral vector selected from the group consisting of a lentiviral vector, a retroviral vector, transposons, cosmids, an adenoviral vector, and an adeno-associated viral (AAV) vector.

### Compositions

Also provided herein are compositions (e.g., pharmaceutical compositions) that include any of the T reg cells (e.g., any of the T reg cells described herein including any of the T reg cells produced using any of the methods described herein) or any of the nucleic acid constructs described herein. In some embodiments, the pharmaceutical compositions can be formulated for intravenous administration. In some embodiments, the pharmaceutical compositions can include a pharmaceutically acceptable carrier (e.g., phosphate buffered saline).

### Kits

Also provided herein are kits that include any of the compositions described herein. For example, a kit can include one or more of any of the nucleic acid constructs described herein. In other examples, a kit can include any of the T reg cells described herein or one or more doses of a composition including any of the T reg cells described herein (e.g., any of the T reg cells described herein or any of the T reg cells produced using any of the methods described herein). In some embodiments, a kit can include instructions for performing any of the methods described herein.

### Methods of Reducing the Number of B Cells in a Tissue and Methods of Treating a Subject Having an Autoimmune Disease

Also provided herein are methods of reducing the number of B cells in a tissue in a subject having an autoimmune disease, where the method comprises administering a therapeutically effective amount of any of the T cells described herein to the subject.

Also provided herein are methods of treating a subject having an autoimmune disease, wherein the method comprises administering a therapeutically effective amount of the T reg cells described herein to the subject.

In some embodiments, the B cell related autoimmune disease is selected from the group of: dermatomyositis, myasthenia gravis, systemic lupus erythematosus, lupus nephritis, Addison's disease, rheumatoid arthritis, multiple sclerosis, Goodpasture's syndrome, Sjogren's syndrome, thyrotoxicosis, scleroderma, chronic active hepatitis, polymyositis/dermatomyositis, polychondritis, pamphigus vulgaris, and amyotrophic lateral sclerosis.

In some embodiments, the subject has previously been treated with a lymphodepletion agent (e.g., cyclophosphamide and/or fludarabine).

Treatment of autoimmune diseases generally requires multiple treatments to control the disease or disorder. Generally, the most common treatments are corticosteroids and cytotoxic drugs, which can be very toxic. These drugs can also suppress the entire immune system, result in serious infection, and have adverse side effects on bone marrow, liver, and/or kidneys. Thus, they do not lend themselves to repeated use (e.g., multiple treatments). In contrast, the use of T cells described herein permits repeated treatments without significant side effects because of its low toxicity profile and greatly reduced side effects.

In some embodiments of any of the methods described herein, the administering comprises administering two or more doses of the T cells. In some embodiments, the administering comprises administering five or more doses of the T cells. In some embodiments, the administering comprises administering ten or more doses of the T cells.

In some embodiments, the methods described herein can further include, prior to the administering step, a step of generating T reg cells. In some embodiments, the step of generating the T reg cells comprises introducing into a T cell a nucleic acid construct comprising a first sequence encoding the humanized chimeric antigen receptor. In some embodiments, the nucleic acid construct further comprises a promoter operably linked to the sequence encoding the chimeric antigen receptor (e.g., humanized chimeric antigen receptor).

In some embodiments, the step of generating the T reg cells further comprises introducing into the T cell a second sequence encoding a FOXP3 polypeptide.

In some embodiments, the first sequence is positioned 5′ relative the second sequence in the nucleic acid construct. In some embodiments, the nucleic acid construct further comprises an additional sequence between the first sequence and the second sequence, wherein the additional sequence operably links the second sequence to the first sequence.

In some embodiments, the second sequence is 5′ positioned relative to the first sequence in the nucleic acid construct. In some embodiments, the nucleic acid construct further comprises an additional sequence between the second sequence and the first sequence, wherein the additional sequence operably links the first sequence to the second sequence.

In some embodiments, the additional sequence comprises an internal ribosome entry site (IRES) sequence or encodes a self-cleaving amino acid. In some embodiments, the additional sequence comprises one or both of a promoter or an enhancer.

In some embodiments, the nucleic acid construct comprises a viral vector selected from the group consisting of a lentiviral vector, a retroviral vector, transposons, cosmids, an adenoviral vector, and an adeno-associated viral (AAV) vector. In some embodiments, the introducing comprises viral transduction.

In some embodiments, the step of further generating the T reg cells further comprises, prior the step of introducing the nucleic acid construct into the T cell, a step of obtaining the T cell from the subject or obtaining the T cell from an allogeneic subject.

In some embodiments, the step of generating the T reg cells further comprises contacting the T cell with an effective amount of one or more CD3-stimulation agents in the absence of a CD28 stimulating agent for a first period of time under conditions that allow for the stimulation of the T cell.

In some embodiments, the T reg cells are administered using parenteral administration (e.g., intravenous administration). In some embodiments, the administering results in amelioration of one or more symptoms of the autoimmune disease in the subject.

In some embodiments, the administering results in a reduction in the number, severity, or frequency of one or more symptoms of the autoimmune diseases in the subject (e.g., as compared to the number, severity, or frequency of the one or more symptoms of the autoimmune disease in the subject prior to treatment). For example, a subject having an autoimmune disease having been administered a T reg cell as described here can experience a reduction in inflammation or autoantibody production.

A pharmaceutical composition containing the T reg cells and a pharmaceutically acceptable carrier or buffer can be administered to the subject (e.g., a human) having an autoimmune disease. For example, a pharmaceutical composition (e.g., the T reg cell along with a pharmaceutically acceptable carrier) to be administered to the subject having an autoimmune disease can be formulated in an injectable form (e.g., s solution and/or suspension). In some embodiments, a pharmaceutical composition containing the T reg cells can include phosphate buffered saline.

Pharmaceutically acceptable carriers, fillers, and vehicles that can be used in a pharmaceutical composition described herein can include, without limitation, ion exchangers, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, and sodium chloride.

Effective dosage can vary depending on the severity of the autoimmune disease, the route of administration, the age and general health condition of the subject, excipient usage, the possibility of co-usage with other therapeutic treatments, and the judgment of the treating physician. An effective amount of a T cell can be any amount that reduces inflammation and autoantibody production within a subject having an autoimmune disease without producing significant toxicity to the subject. In some cases, the T cells can be a purified population of T reg cells generated as described herein. In some cases, the purity of the population of T reg cells can be assessed using any appropriate method, including, without limitation, flow cytometry. In some cases, the population of T cells to be administered can include a range of purities from about 70% to about 100%, from about 70% to about 90%, from about 70% to about 80%, from about 80% to about 90%, from about 90% to about 100%, from about 80% to about 100%, from about 80% to about 90%, or from about 90% to 100%. In some cases, the dosage (e.g., number of T reg cells to be administered) can adjusted based on the level of purity of the T reg cells.

The frequency of administration of a T cell can be any frequency that reduces inflammation or autoantibody production within a subject having an autoimmune disease without producing toxicity to the subject. In some cases, the actual frequency of administration can vary depending on various factors including, without limitation, the effective amount, duration of treatment, use of multiple treatment agents, route of administration, and severity of the condition may require an increase or decrease in frequency of administration.

An effective duration for administering a composition containing the anti-CD19 CAR T cell construct can be any duration that reduces inflammation or autoantibody production within the subject having an autoimmune disease without producing toxicity to the subject. In some cases, the effective duration can vary from several days to several months. In general, the effective treatment duration for administering a composition containing the T cell to treat an autoimmune disease can range in duration from about one month to about five years (e.g., from about two months to about five years, from about three months to about five years, from about six months to about five years, from about eight months to about five years, from about one year to about five years, from about one month to about four years, from about one month to about three years, from about one month to about two years, from about six months to about four years, from about six months to about three years, or from about six months to about two years).

In some cases, a course of treatment and/or the severity of one or more symptoms related to autoimmune disease can be monitored. Any appropriate method can be used to determine whether the autoimmune disease is being treated. For example, immunological techniques (e.g., ELISA) can be performed to determine if the level of autoantibodies present within the subject being treated as described herein is reduced following the administration of the T reg cells. Remission and relapse of the disease can be monitored by testing for one or more markers of the autoimmune disease.

Any appropriate autoimmune disease or disorder can be treated with a T reg cell (e.g., T reg cell) as described herein. In some cases, an autoimmune disease or disorder is caused by the accumulation of autoantibodies and can be treated with a T reg cell as described herein.

For example, a subject can receive 2, 3, 4, 5, 6, 7, 8, 9, 10, 11, 12, 13, 14, 15, 16, 17, 18, 19, 20, 30, 40, 50, or more doses of any of the T reg cells described herein. In some embodiments, a subject receives at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 doses of a T reg cell (e.g., a T reg cell expressing a chimeric antigen receptor comprising SEQ ID NO: 14).

In some embodiments, the method result in a reduction (e.g., at least a 1% reduction, at least a 5% reduction, at least a 10% reduction, at least a 15% reduction, at least a 20% reduction, at least a 25% reduction, at least a 30% reduction, at least a 35% reduction, at least a 40% reduction, at least a 45% reduction, at least a 50% reduction, at least a 55% reduction, at least a 60% reduction, at least a 65% reduction, at least a 70% reduction, at least a 75% reduction, at least a 80% reduction, at least a 85% reduction, at least a 90% reduction, at least a 95% reduction, or at least a 99% reduction, or about a 1% reduction to about a 99% reduction, about a 1% reduction to about a 90% reduction, about a 1% reduction to about a 80% reduction, about a 1% reduction to about a 70% reduction, about a 1% reduction to about a 60% reduction, about a 1% reduction to about a 50% reduction, about a 1% reduction to about a 45% reduction, about a 1% reduction to about a 40% reduction, about a 1% reduction to about a 35% reduction, about a 1% reduction to about a 30% reduction, about a 1% reduction to about a 25% reduction, about a 1% reduction to about a 20% reduction, about a 1% reduction to about a 15% reduction, about a 1% reduction to about a 10% reduction, about a 1% reduction to about a 5% reduction, about a 5% reduction to about a 99% reduction, about a 5% reduction to about a 90% reduction, about a 5% reduction to about a 80% reduction, about a 5% reduction to about a 70% reduction, about a 5% reduction to about a 60% reduction, about a 5% reduction to about a 50% reduction, about a 5% reduction to about a 45% reduction, about a 5% reduction to about a 40% reduction, about a 5% reduction to about a 35% reduction, about a 5% reduction to about a 30% reduction, about a 5% reduction to about a 25% reduction, about a 5% reduction to about a 20% reduction, about a 5% reduction to about a 15% reduction, about a 5% reduction to about a 10% reduction, about a 10% reduction to about a 99% reduction, about a 10% reduction to about a 90% reduction, about a 10% reduction to about a 80% reduction, about a 10% reduction to about a 70% reduction, about a 10% reduction to about a 60% reduction, about a 10% reduction to about a 50% reduction, about a 10% reduction to about a 45% reduction, about a 10% reduction to about a 40% reduction, about a 10% reduction to about a 35% reduction, about a 10% reduction to about a 30% reduction, about a 10% reduction to about a 25% reduction, about a 10% reduction to about a 20% reduction, about a 10% reduction to about a 15% reduction, about a 15% reduction to about a 99% reduction, about a 15% reduction to about a 90% reduction, about a 15% reduction to about a 80% reduction, about a 15% reduction to about a 70% reduction, about a 15% reduction to about a 60% reduction, about a 15% reduction to about a 50% reduction, about a 15% reduction to about a 45% reduction, about a 15% reduction to about a 40% reduction, about a 15% reduction to about a 35% reduction, about a 15% reduction to about a 30% reduction, about a 15% reduction to about a 25% reduction, about a 15% reduction to about a 20% reduction, about a 20% reduction to about a 99% reduction, about a 20% reduction to about a 90% reduction, about a 20% reduction to about a 80% reduction, about a 20% reduction to about a 70% reduction, about a 20% reduction to about a 60% reduction, about a 20% reduction to about a 50% reduction, about a 20% reduction to about a 45% reduction, about a 20% reduction to about a 40% reduction, about a 20% reduction to about a 35% reduction, about a 20% reduction to about a 30% reduction, about a 20% reduction to about a 25% reduction, about a 25% reduction to about a 99% reduction, about a 25% reduction to about a 90% reduction, about a 25% reduction to about a 80% reduction, about a 25% reduction to about a 70% reduction, about a 25% reduction to about a 60% reduction, about a 25% reduction to about a 50% reduction, about a 25% reduction to about a 45% reduction, about a 25% reduction to about a 40% reduction, about a 25% reduction to about a 35% reduction, about a 25% reduction to about a 30% reduction, about a 30% reduction to about a 99% reduction, about a 30% reduction to about a 90% reduction, about a 30% reduction to about a 80% reduction, about a 30% reduction to about a 70% reduction, about a 30% reduction to about a 60% reduction, about a 30% reduction to about a 50% reduction, about a 30% reduction to about a 45% reduction, about a 30% reduction to about a 40% reduction, about a 30% reduction to about a 35% reduction, about a 35% reduction to about a 99% reduction, about a 35% reduction to about a 90% reduction, about a 35% reduction to about a 80% reduction, about a 35% reduction to about a 70% reduction, about a 35% reduction to about a 60% reduction, about a 35% reduction to about a 50% reduction, about a 35% reduction to about a 45% reduction, about a 35% reduction to about a 40% reduction, about a 40% reduction to about a 99% reduction, about a 40% reduction to about a 90% reduction, about a 40% reduction to about a 80% reduction, about a 40% reduction to about a 70% reduction, about a 40% reduction to about a 60% reduction, about a 40% reduction to about a 50% reduction, about a 40% reduction to about a 45% reduction, about a 45% reduction to about a 99% reduction, about a 45% reduction to about a 90% reduction, about a 45% reduction to about a 80% reduction, about a 45% reduction to about a 70% reduction, about a 45% reduction to about a 60% reduction, about a 45% reduction to about a 50% reduction, about a 50% reduction to about a 99% reduction, about a 50% reduction to about a 90% reduction, about a 50% reduction to about a 80% reduction, about a 50% reduction to about a 70% reduction, about a 50% reduction to about a 60% reduction, about a 60% reduction to about a 99% reduction, about a 60% reduction to about a 90% reduction, about a 60% reduction to about a 80% reduction, about a 60% reduction to about a 70% reduction, about a 70% reduction to about a 99% reduction, about a 70% reduction to about a 90% reduction, about a 70% reduction to about a 80% reduction, about a 80% reduction to about a 99% reduction, about a 80% reduction to about a 90% reduction, or about a 90% reduction to about a 99% reduction) in the number of B cells in a tissue of the subject having an autoimmune disease, e.g., as compared to the levels in the subject prior to treatment or the levels in a similar subject not treated or receiving a different treatment.

In some embodiments, the methods described herein result in a reduction (e.g., at least a 1% reduction, at least a 5% reduction, at least a 10% reduction, at least a 15% reduction, at least a 20% reduction, at least a 25% reduction, at least a 30% reduction, at least a 35% reduction, at least a 40% reduction, at least a 45% reduction, at least a 50% reduction, at least a 55% reduction, at least a 60% reduction, at least a 65% reduction, at least a 70% reduction, at least a 75% reduction, at least a 80% reduction, at least a 85% reduction, at least a 90% reduction, at least a 95% reduction, or at least a 99% reduction, or about a 1% reduction to about a 99% reduction (or any of the subranges of this range described herein) in the level of autoantibodies in the subject having the autoimmune disease, e.g., as compared to the levels in the subject prior to treatment or the levels in a similar subject not treated or receiving a different treatment.

The invention will be further described in the following examples, which do not limit the scope of the invention described in the claims.

### EXAMPLES

### Example 1. Preparation of T Cells Comprising Hu19-CD828Z

Hu19-CD828Z is prepared as described in U.S. Pat. No. 10,287,350. Briefly, fully human anti-CD19 CARs were generated by utilizing sequences of the fully human 47G4 monoclonal antibody (described in U.S. Patent Application Publication No. 2010/0104509). The 47G4 antibody was generated by vaccinating mice of the KM strain, which carry a human kappa light chain transgene and a human heavy chain transchromosome. The sequences of the 47G4 antibody light chain and heavy chain variable regions were obtained from U.S. Patent Application Publication No. 2010/0104509. A 47G4 scFv was designed comprising the following elements from 5′ to 3′: a CD8 signal sequence, the 47G4 antibody light chain variable region, a linker peptide comprising the amino acid sequence GSTSGSGKPGSGEGSTKG (SEQ ID NO: 9) (see Cooper et al., Blood, 101 (4): 1637-1644 (2003)), and the 47G4 antibody heavy chain variable region. A DNA sequence encoding a CAR was then designed comprising the following components from 5′ to 3′: the 47G4 scFv described above, part of the extracellular region and all of the transmembrane region of the human CD8 molecule, and the cytoplasmic portions of the human CD28 molecule and the human CD3 zeta. molecule. This CAR was designated 47G4-CD828Z (SEQ ID NO: 14), and the sequence was synthesized by Invitrogen (Carlsbad, Calif.).

Further, a set of experiments is performed to assess the effect of expression of a Hu19-828Z polypeptide. In these experiments, CD4 T cells are transduced with a lentivirus where the lentiviral vector includes a nucleic acid sequence encoding Hu19-CD828Z polypeptide. The vector includes an EF1α promoter. Lentivirus is produced in HEK293 cells according to standard protocols.

Alternatively, a set of experiments is performed to assess the effect of co-expression of a Hu19-CD828Z polypeptide and a FOXP3 polypeptide. In these experiments, CD4+ T cells are transduced with a lentivirus where the lentiviral vector includes a first nucleic acid sequence encoding a FOXP3 polypeptide harboring mutations in NES1 and NES2 that result in nuclear localization of FOXP3 and a second nucleic acid sequence encoding Hu19-CD828Z polypeptide. The vector includes an EF1α promoter. Lentivirus is produced in HEK293 cells according to standard protocols.

CD4 T cells are counted and checked for viability. Next cells are re-suspended in fresh serum free ImmunoCult T cell expansion media at a concentration of 10⁶ cells/mL. Then 500 TL (˜500,000 cells) of the cell suspension is aliquoted to each well. The cells are then cultured in the presence of CD3/CD28 for 1-2 days prior to addition of virus. Different concentrations of lentiviral particles are added to each well for the desired target MOI. The plates are then sealed with parafilm, and the cells are spun in a table top centrifuge at 300×g for 5 minutes. After spinoculation, the cells are incubated at 37° C. The cells are then assessed for Hu19-CD828Z expression, FOXP3 expression (if co-transduced) and cellular localization, and expression of a T reg phenotype.

### Example 2. Use of Hu19-CD828Z to Treat Lupus Nephritis

An effective amount of therapeutic T cells introduced (e.g., transduced, etc.) with a vector encoding the CAR of Example 1, with or without co-expression of FOXP3, is administered to a subject with lupus nephritis. The dose is in the range of 0.3×10⁸ CAR+ viable T cells, with about 3-fold escalation up to 3×10⁸ CAR+ viable T cells. In some cases, a course of treatment and/or the severity of one or more symptoms related to autoimmune disease or disorder can be monitored. Any appropriate method can be used to determine whether the autoimmune disease or disorder is being treated. For example, immunological techniques (e.g., ELISA) can be performed to determine if the level of autoantibodies present within a subject being treated as described herein is reduced following the administration of the T cells. Remission and relapse of the disease can be monitored by testing for one or more markers of autoimmune disease or disorder. Depending on the presence of autoantibodies, the patient may be retreated one or more times until the autoantibodies are not detectable.

### Example 3. In Vitro Assessment of Systemic Lupus Erythematosus (SLE) Patient-Derived Hu19-CD828Z Transduced Cells

Cytolytic activity and activation of Hu19-CD828Z generated from SLE patient PBMCs against human CD19⁺ malignant B cell cancer cells and autologous SLE patient primary B cells were assessed.

Briefly, three lots of Hu19-CD828Z cells were generated from SLE patient peripheral blood mononuclear cells (PBMCs), two lots of Hu 19-CD828Z cells were generated from healthy donor (HD) PBMCs, or untransduced T cells from the same donors were co-cultured overnight with target cells. Target cells were either the human ALL cell line NALM6 (known to express high levels of CD19), autologous (i.e., donor matched) primary B cells that express CD19, and/or the human chronic myeloid leukemia (CML) cell line K562 that does not express CD1, at Effector:Target (E:T) ratios ranging from 0:1 to 3:1 for NALM6 and K562 and 0:5 to 10:1 for autologous primary B cell co-cultures.

Effector and target cells alone were included as negative controls. After co-incubation, the amount of cytokines in the supernatant and the number of live target cells were quantitated.

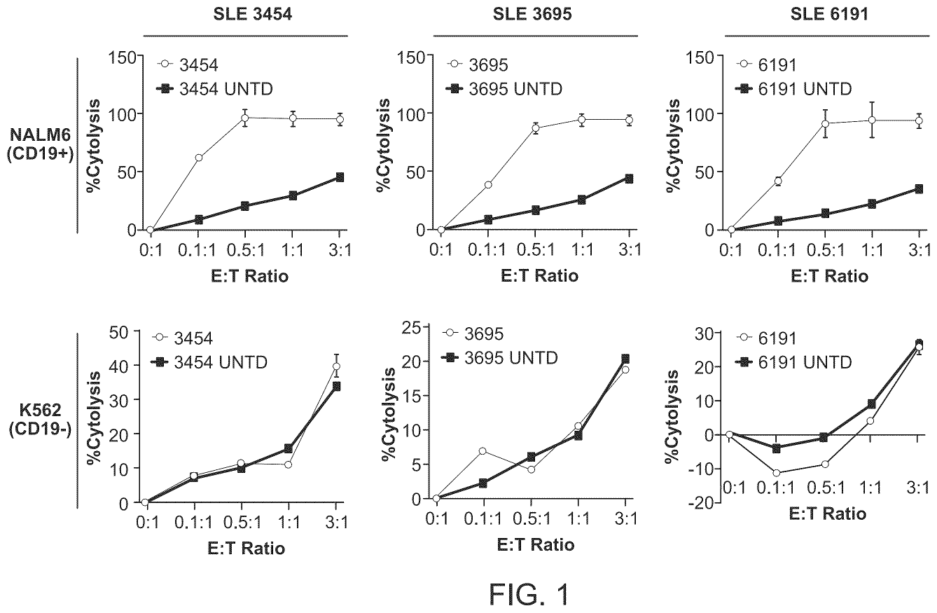

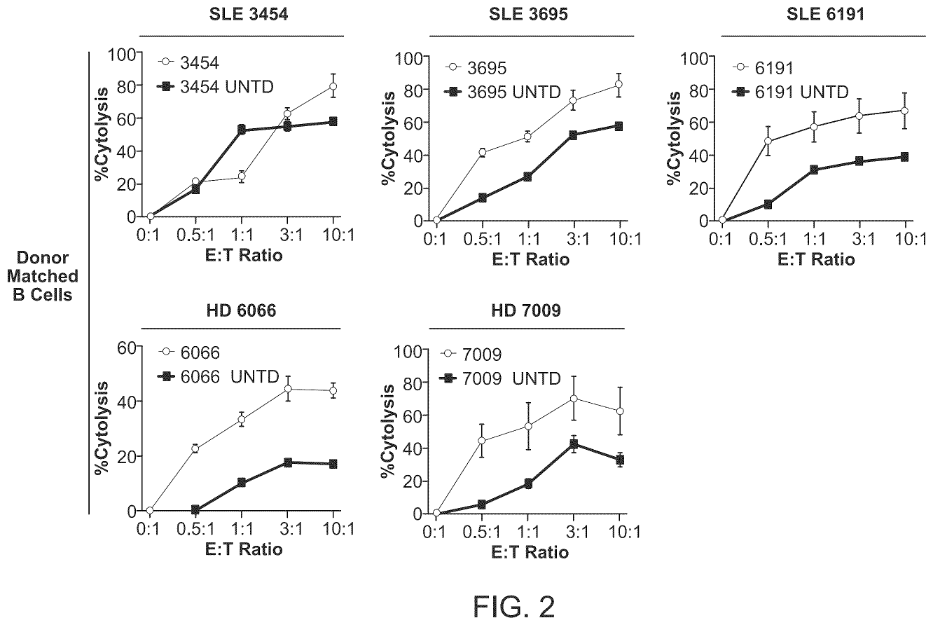

SLE-derived Hu19-CD828Z cells showed CAR-mediated cytotoxicity, cytokine release, and proliferation in a CD19-dependent manner. Cytotoxicity was demonstrated by robust and dose-dependent cytotoxicity against both a CD19⁺ human B cell cancer cell line (NALM6) and minimal cytotoxicity against the K562 CD19⁻ cell line (FIG. 1). SLE-derived Hu19-CD828Z cells from two donors (3695 and 6191) induced strong, dose-dependent cytotoxicity of autologous B cells with the level of cytotoxicity being significantly greater than observed for untransduced T cells, thereby demonstrating that the difference in cytotoxicity is driven by the CAR (FIG. 2).

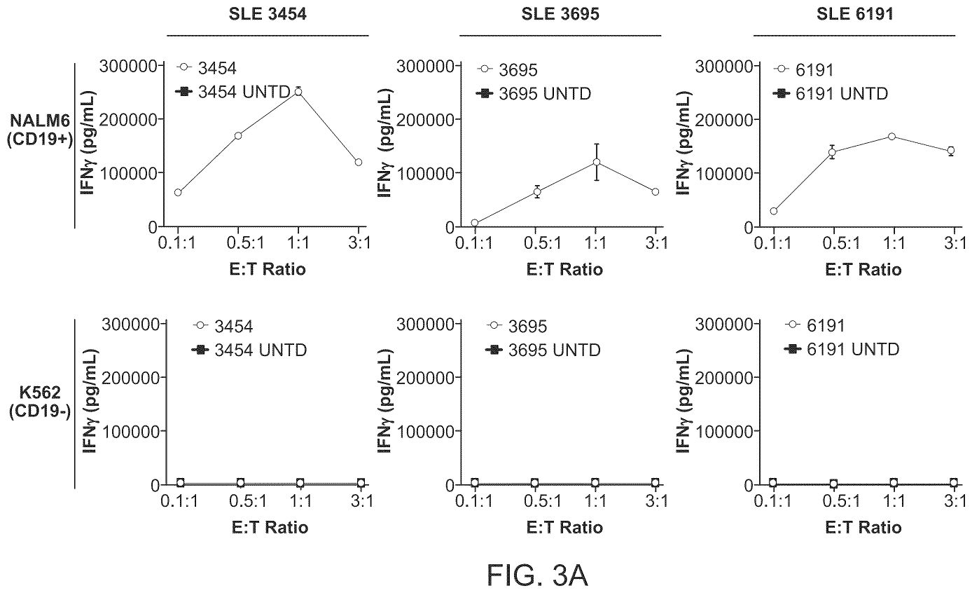

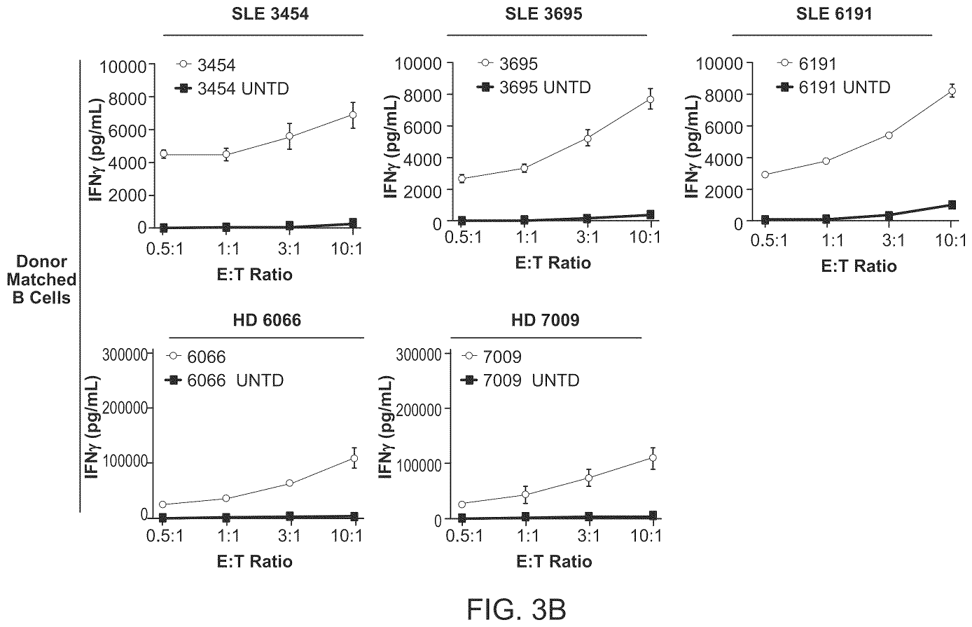

All three lots of SLE-derived Hu19-CD828Z cells also showed CD19-mediated and dose-dependent production of Granzyme B and the cytokines IL-10, IL-13, IL-2, IL-4, IL-8, and TNFα, which are directly related to T cell activation and/or T cell-mediated cytotoxicity, upon Hu19-CCD828Z co-culture with CD19⁺ NALM6 cells and autologous B cells, but not with the CD19⁻K562 cells (data not shown). Specifically, interferon gamma (IFNγ) was the predominant cytokine detected as shown in FIGS. 3A and 3B.

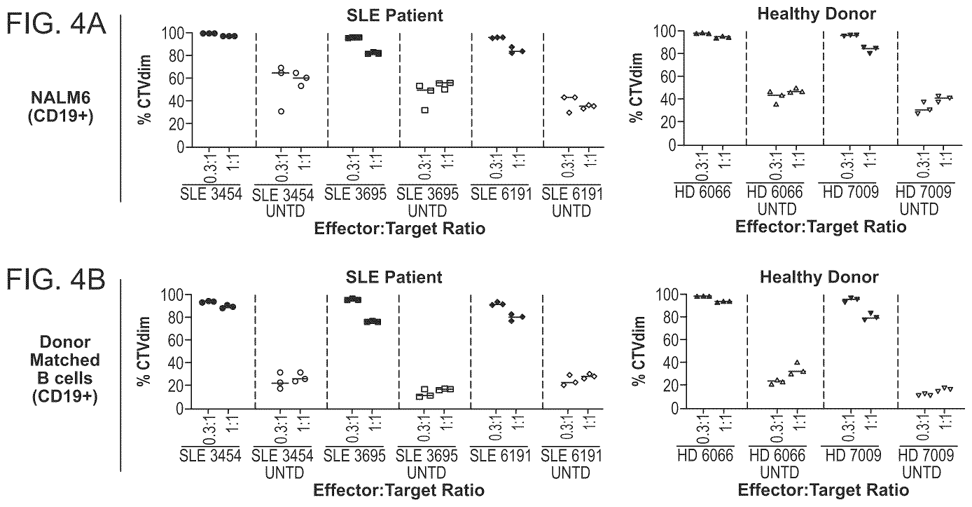

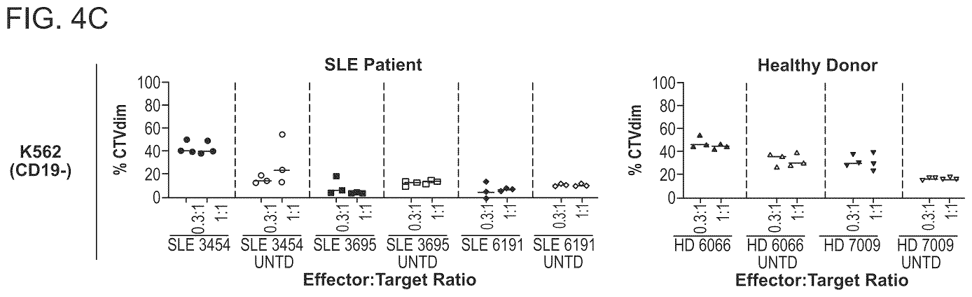

For the target-dependent, CAR-mediated proliferation studies, the same set of SLE- and HD-derived Hu19-CD828Z and untransduced T cells were co-cultured for 96 hours with the same set of CD19⁺ and CD19⁻ target cells (as described herein). Effector and target cells alone were included as negative controls. After co-incubation, the number of Cell Trace Violet (CTV) dim proliferating effector cells was quantified. CD19 was confirmed to be expressed on NALM6 and autologous primary B cells but not expressed on K562 cells (data not shown). SLE-derived Hu19-CD828Z cells also showed CAR-mediated and CD19-mediated proliferation comparable to that from HD-derived Hu19-CD828Z cells (FIGS. 4A, 4B, and 4C).

The data demonstrate that SLE patient-derived Hu19-CD828Z transduced PBMCs exhibit CAR-mediated and CD19-dependent cytotoxicity, cytokine release, and cellular proliferation as demonstrated by activity during co-culture with CD19⁺ cancer cells (NALM6 cells) and autologous primary B cells, but not control CD19⁻ cells (K562). The results from the cytokine release (FIGS. 3A-B) and proliferation assay (FIGS. 4A-4C) showed clear target-mediated response demonstrating that the activity of the Hu19-CD828Z CAR construct in SLE and Healthy Donor-derived samples is comparable.

### Example 4. Use of Hu19-CD828Z to Treat Systemic Lupus Erythematosus

An effective amount of therapeutic T cells introduced (e.g., transduced, etc.) with a vector encoding the CAR of Example 1, with or without co-expression of FOXP3, is administered to a subject with Systemic Lupus Erythematosus. The dose is in the range of 0.3×10⁸ CAR⁺ viable T cells, with about 3-fold escalation up to 3×10⁸ CAR⁺ viable T cells. In some cases, a course of treatment and/or the severity of one or more symptoms related to autoimmune disease or disorder can be monitored. Any appropriate method can be used to determine whether the autoimmune disease or disorder is being treated. For example, immunological techniques (e.g., ELISA) can be performed to determine if the level of autoantibodies present within a subject being treated as described herein is reduced following the administration of the T cells. Remission and relapse of the disease can be monitored by testing for one or more markers of autoimmune disease or disorder. Depending on the presence of autoantibodies, the patient may be retreated one or more times until the autoantibodies are not detectable.

### Example 5. Use of Hu19-CD828Z to Treat Rheumatoid Arthritis

An effective amount of therapeutic cells, in the range of 0.3×10⁸ CAR⁺ viable T cells, with about 3-fold escalation up to 3×10⁸ CAR⁺ viable T cells introduced (e.g., transduced, etc.) with a vector encoding the CAR of Example 1, with or without co-expression of FOXP3, is administered to a subject with rheumatoid arthritis. In some cases, a course of treatment and/or the severity of one or more symptoms related to autoimmune disease or disorder can be monitored. Any appropriate method can be used to determine whether the autoimmune disease or disorder is being treated. For example, immunological techniques (e.g., ELISA) can be performed to determine if the level of autoantibodies present within a subject being treated as described herein is reduced following the administration of the T cells. Remission and relapse of the disease can be monitored by testing for one or more markers of autoimmune disease or disorder.

### Example 6. Use of Hu19-CD828Z to Treat Sjogren's Syndrome

An effective amount of therapeutic cells, in the range of 0.3×10⁸ CAR+ viable T cells, with about 3-fold escalation up to 3×10⁸ CAR+ viable T cells introduced (e.g., transduced, etc.) with a vector encoding the CAR of Example 1, with or without co-expression of FOXP3, is administered to a subject with Sjogren's syndrome. In some cases, a course of treatment and/or the severity of one or more symptoms related to autoimmune disease or disorder can be monitored. Any appropriate method can be used to determine whether the autoimmune disease or disorder is being treated. For example, immunological techniques (e.g., ELISA) can be performed to determine if the level of autoantibodies present within a subject being treated as described herein is reduced following the administration of the T cells. Remission and relapse of the disease can be monitored by testing for one or more markers of autoimmune disease or disorder.

### Example 7. a Method of Treating an Autoimmune Disease According to Examples 2 and 4-6 Following Lymphodepletion with Cyclophosphamide

5 to 7 days prior to administration of Hu19-CD828Z in any one of Examples 2 and 4-6 lymphodepleting chemotherapy using cyclophosphamide (CYC) 300 mg/m² and fludarabine (Flu) 30 mg/m² is administered using standard procedures known in the art.

SEQUENCE APPENDIX

SEQ ID NO: 1 Hu19-CD828Z Heavy Chain CDR1
SYAIS
SEQ ID NO: 2 Hu19-CD828Z Heavy Chain CDR2
GIIPIFGTTNYAQQFQG
SEQ ID NO: 3 Hu19-CD828Z Heavy Chain CDR3
EAVAADWLDP
SEQ ID NO: 4 Hu19-CD828Z Light Chain CDR1
RASQSVSSSYLA
SEQ ID NO: 5 Hu19-CD828Z Light Chain CDR2
GASSRAT
SEQ ID NO: 6 Hu19-CD828Z Light Chain CDR3
QQYGSSRFT
SEQ ID NO: 7 Hu19-CD828Z Heavy Chain Variable
Region
QVQLVQSGAEVKKPGSSVKVSCKDSGGTFSSYAIS
WVRQAPGQGLEWMGGIIPIFGTTNYAQQFQGRVTI
TADESTSTAYMELSSLRSEDTAVYYCAREAVAADW
LDPWGQGTLVTVSS
SEQ ID NO: 8 Hu19-CD828Z Light Chain Variable
Region
EIVLTQSPGTLSLSPGERATLSCRASQSVSSSYLA
WYQQKPGQAPRLLIYGASSRATGIPDRFSGSGSGT
DFTLTISRLEPEDFAVYYCQQYGSSRFTFGPGTKV
DIK
SEQ ID NO: 9 Hu19-CD828Z Peptide Linker
GSTSGSGKPGSGEGSTKG
SEQ ID NO: 10 CD3 zeta cytoplasmic signaling
domain
MKWKALFTAAILQAQLPITEAQSFGLLDPKLCYLL
DGILFIYGVILTALFLRVKFSRSADAPAYQQGQNQ
LYNELNLGRREEYDVLDKRRGRDPEMGGKPQRRKN
PQEGLYNELQKDKMAEAYSEIGMKGERRRGKGHDG
LYQGLSTATKDTYDALHMQALPPR
SEQ ID NO: 11 CD28 co-stimulatory domain
IEVMYPPPYLDNEKSNGTIIHVKGKHLCPSPLFPG
PSKPFWVLVVVGGVLACYSLLVTVAFIIFWVRSKR
SRLLHSDYMNMTPRRPGPTRKHYQPYAPPRDFAAY
SEQ ID NO: 12 CD28 transmembrane domain
FWVLVVVG GVLACYSLLV TVAFIIFWV
SEQ ID NO: 13 FOXP3 nucleotide sequence
AGTTTCCCACAAGCCAGGCTGATCCTTTTCTGTCA
GTCCACTTCACCAAGCCTGCCCTTGGACAAGGACC
CGATGCCCAACCCCAGGCCTGGCAAGCCCTCGGCC
CCTTCCTTGGCCCTTGGCCCATCCCCAGGAGCCTC
GCCCAGCTGGAGGGCTGCACCCAAAGCCTCAGACC
TGCTGGGGGCCCGGGGCCCAGGGGGAACCTTCCAG
GGCCGAGATCTTCGAGGCGGGGCCCATGCCTCCTC
TTCTTCCTTGAACCCCATGCCACCATCGCAGCTGC
AGCTCTCAACGGTGGATGCCCACGCCCGGACCCCT
GTGCTGCAGGTGCACCCCCTGGAGAGCCCAGCCAT
GATCAGCCTCACACCACCCACCACCGCCACTGGGG
TCTTCTCCCTCAAGGCCCGGCCTGGCCTCCCACCT
GGGATCAACGTGGCCAGCCTGGAATGGGTGTCCAG
GGAGCCGGCACTGCTCTGCACCTTCCCAAATCCCA
GTGCACCCAGGAAGGACAGCACCCTTTCGGCTGTG
CCCCAGAGCTCCTACCCACTGCTGGCAAATGGTGT
CTGCAAGTGGCCCGGATGTGAGAAGGTCTTCGAAG
AGCCAGAGGACTTCCTCAAGCACTGCCAGGCGGAC
CATCTTCTGGATGAGAAGGGCAGGGCACAATGTCT
CCTCCAGAGAGAGATGGTACAGTCTCTGGAGCAGC
AGCTGGTGCTGGAGAAGGAGAAGCTGAGTGCCATG
CAGGCCCACCTGGCTGGGAAAATGGCACTGACCAA
GGCTTCATCTGTGGCATCATCCGACAAGGGCTCCT
GCTGCATCGTAGCTGCTGGCAGCCAAGGCCCTGTC
GTCCCAGCCTGGTCTGGCCCCCGGGAGGCCCCTGA
CAGCCTGTTTGCTGTCCGGAGGCACCTGTGGGGTA
GCCATGGAAACAGCACATTCCCAGAGTTCCTCCAC
AACATGGACTACTTCAAGTTCCACAACATGCGACC
CCCTTTCACCTACGCCACGCTCATCCGCTGGGCCA
TCCTGGAGGCTCCAGAGAAGCAGCGGACACTCAAT
GAGATCTACCACTGGTTCACACGCATGTTTGCCTT
CTTCAGAAACCATCCTGCCACCTGGAAGAACGCCA
TCCGCCACAACCTGAGTCTGCACAAGTGCTTTGTG
CGGGTGGAGAGCGAGAAGGGGGCTGTGTGGACCGT
GGATGAGCTGGAGTTCCGCAAGAAACGGAGCCAGA
GGCCCAGCAGGTGTTCCAACCCTACACCTGGCCCC
TGACCTCAAGATCAAGGAAAGGAGGATGGACGAAC
AGGGGCCAAACTGGTGGGAGGCAGAGGTGGTGGGG
GCAGGGATGATAGGCCCTGGATGTGCCCACAGGGA
CCAAGAAGTGAGGTTTCCACTGTCTTGCCTGCCAG
GGCCCCTGTTCCCCCGCTGGCAGCCACCCCCTCCC
CCATCATATCCTTTGCCCCAAGGCTGCTCAGAGGG
GCCCCGGTCCTGGCCCCAGCCCCCACCTCCGCCCC
AGACACACCCCCCAGTCGAGCCCTGCAGCCAAACA
GAGCCTTCACAACCAGCCACACAGAGCCTGCCTCA
GCTGCTCGCACAGATTACTTCAGGGCTGGAAAAGT
CACACAGACACACAAAATGTCACAATCCTGTCCCT
CACTCAACACAAACCCCAAAACACAGAGAGCCTGC
CTCAGTACACTCAAACAACCTCAAAGCTGCATCAT
CACACAATCACACACAAGCACAGCCCTGACAACCC
ACACACCCCAAGGCACGCACCCACAGCCAGCCTCA
GGGCCCACAGGGGCACTGTCAACACAGGGGTGTGC
CCAGAGGCCTACACAGAAGCAGCGTCAGTACCCTC
AGGATCTGAGGTCCCAACACGTGCTCGCTCACACA
CACGGCCTGTTAGAATTCACCTGTGTATCTCACGC
ATATGCACACGCACAGCCCCCCAGTGGGTCTCTTG
AGTCCCGTGCAGACACACACAGCCACACACACTGC
CTTGCCAAAAATACCCCGTGTCTCCCCTGCCACTC
ACCTCACTCCCATTCCCTGAGCCCTGATCCATGCC
TCAGCTTAGACTGCAGAGGAACTACTCATTTATTT
GGGATCCAAGGCCCCCAACCCACAGTACCGTCCCC
AATAAACTGCAGCCGAGCTCCCCA
SEQ ID NO: 14 Full-length Hu19-CD828Z Chimeric
Antigen Receptor
MALPVTALLLPLALLLHAARPEIVLTQSPGTLSLS
PGERATLSCRASQSVSSSYLAWYQQKPGQAPRLLI
YGASSRATGIPDRFSGSGSGTDFTLTISRLEPEDF
AVYYCQQYGSSRFTFGPGTKVDIKGSTSGSGKPGS
GEGSTKGQVQLVQSGAEVKKPGSSVKVSCKDSGGT
FSSYAISWVRQAPGQGLEWMGGIIPIFGTTNYAQQ
FQGRVTITADESTSTAYMELSSLRSEDTAVYYCAR
EAVAADWLDPWGQGTLVTVSSFVPVFLPAKPTTTP
APRPPTPAPTIASQPLSLRPEACRPAAGGAVHTRG
LDFACDIYIWAPLAGTCGVLLLSLVITLYCNHRNR
SKRSRLLHSDYMNMTPRRPGPTRKHYQPYAPPRDF
AAYRSRVKFSRSADAPAYQQGQNQLYNELNLGRRE
EYDVLDKRRGRDPEMGGKPRRKNPQEGLYNELQKD
KMAEAYSEIGMKGERRRGKGHDGLYQGLSTATKDT
YDALHMQALPPR
SEQ ID NO: 15 Human FOXP3 Protein
MPNPRPGKPSAPSLALGPSPGASPSWRAAPKASDL
LGARGPGGTFQGRDLRGGAHASSSSLNPMPPSQLQ
LSTVDAHARTPVLQVHPLESPAMISLTPPTTATGV
FSLKARPGLPPGINVASLEWVSREPALLCTFPNPS
APRKDSTLSAVPQSSYPLLANGVCKWPGCEKVFEE
PEDFLKHCQADHLLDEKGRAQCLLQREMVQSLEQQ
LVLEKEKLSAMQAHLAGKMALTKASSVASSDKGSC
CIVAAGSQGPVVPAWSGPREAPDSLFAVRRHLWGS
HGNSTFPEFLHNMDYFKFHNMRPPFTYATLIRWAI
LEAPEKQRTLNEIYHWFTRMFAFFRNHPATWKNAI
RHNLSLHKCFVRVESEKGAVWTVDELEFRKKRSQR
PSRCSNPTPGP

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of engineering a non-toxic T cell of a human subject having an autoimmune disease, the method comprising introducing to the T cell at least one nucleic acid encoding a chimeric antigen receptor.

2. The method of claim 1, wherein the chimeric antigen receptor comprises an extracellular antigen-binding domain, a transmembrane domain, and an intracellular domain, wherein the intracellular domain comprises a cytoplasmic signaling domain and one or more co-stimulatory domains.

3. The method of claim 2, wherein the extracellular antigen-binding domain is capable of binding to CD19, the transmembrane domain comprises a human CD8 transmembrane domain, the cytoplasmic signaling domain comprises a human CD3 zeta intracellular signaling domain, and the one or more co-stimulatory domain comprises an intracellular signaling domain from human CD28.

4. The method of claim 3, wherein the extracellular antigen-binding domain comprises an antigen-binding fragment of a fully human antibody.

5. The method of claim 4, wherein the antigen-binding fragment is selected from the group consisting of a Fab, a F(ab′) 2 fragment, a scFv, a scAb, a dAb, and a single domain antibody.

6. The method of claim 3, wherein the extracellular antigen-binding domain comprises:
(i) a heavy chain variable domain comprising a CDR1, a CDR2, and a CDR3 of SEQ ID NOs: 1-3, respectively; and
(ii) a light chain variable domain comprising a CDR1, a CDR2, and a CDR3 of SEQ ID NOs: 4-6, respectively.

7. The method of claim 6, wherein the extracellular antigen-binding domain comprises:
(i) a heavy chain variable domain that is at least 90% identical to SEQ ID NO: 7; and
(ii) a light chain variable domain that is at least 90% identical to SEQ ID NO: 8.

8. The method of claim 1, wherein the chimeric antigen receptor comprises a sequence that is at least 90% identical to SEQ ID NO: 14.

9. The method of claim 8, wherein the chimeric antigen receptor comprises a sequence that is at least 95% identical to SEQ ID NO: 14.

10. The method of claim 9, wherein the chimeric antigen receptor comprises SEQ ID NO: 14.

11. The method of claim 1, wherein the autoimmune disease is a B cell related autoimmune disease.

12. The method of claim 11, wherein the B cell related autoimmune disease is rheumatoid arthritis, Sjogren's syndrome, lupus nephritis, or systemic lupus erythematosus.

13. The method of claim 12, wherein the B cell related autoimmune disease is systemic lupus erythematosus.

14-30. (canceled)
